(19) United States
(12) Patent Application Publication
(10) Pub. No.: US 2024/0376152 A1
(43) Pub. Date: Nov. 14, 2024

(54) PEPTIDES THAT BIND TO TEXTILES

(71) Applicant: HENKEL AG & CO. KGAA, Düsseldorf (DE)

(72) Inventors: Shohana ISLAM, Düsseldorf (DE); Iva ANIC, Düsseldorf (DE); Christian KASTNER, Düsseldorf (DE); Irmgard SCHMIDT, Solingen (DE); Roland BREVES, Mettmann (DE); Christian DEGERING, Erkrath (DE); Susanne WIELAND, Zons/Dormagen (DE)

(21) Appl. No.: 18/564,736

(22) PCT Filed: Dec. 7, 2022
(86) PCT No.: PCT/EP2022/084768

§ 371 Date: Nov. 28, 2023

(30) Foreign Application Priority Data
Dec. 16, 2021 (DE) 10 2021 133 434.9

(51) Int. Cl. C07K 7/08; C11D 3/37; C11D 3/386

(52) U.S. Cl. CPC C07K 7/08; C11D 3/3719; C11D 3/386; C11D 2111/12

(57) ABSTRACT

The invention relates to a peptide as described herein. The invention further relates to a detergent or cleaning agent which comprises at least one peptide according to the invention and to the use of a peptide according to the invention and/or a detergent or cleaning agent according to the invention for cleaning and/or treating textiles that are made of plastic or consisting to some extent of plastic.

The invention relates to a peptide as described herein. The invention further relates to a detergent or cleaning agent which comprises at least one peptide according to the invention and to the use of a peptide according to the invention and/or a detergent or cleaning agent according to the invention for cleaning and/or treating textiles that are made of plastic or consisting to some extent of plastic.

Intensive research is being conducted on the development of adhesive peptides. In particular, peptides that specifically bind to or interact with oxidic surfaces such as metal surfaces have already been described (WO 2014/072313 A1). Adhesive peptides are also of interest for many other surfaces that, due to their material or surface properties, make it difficult to treat or attach other objects, fabrics or compounds, for example. In the case of such surfaces, those with low surface energies, such as those often found in certain plastics, are particularly problematic. Such plastics are also referred to in the prior art as low surface energy polymers (LSEP) and comprise, for example, many polyolefins or polymers with ester functions.

The inventors have found and developed special peptides that are surprisingly suitable for adhesion to plastic surfaces, preferably LSEP (low surface energy polymers) surfaces, and in particular textiles made of (LSEP) plastic or with an (LSEP) plastic content, and that partially or completely solve the above problems. These peptides are particularly suitable for use in detergents and cleaning means, and can contribute to improved cleaning performance and/or impart special properties to textiles. Furthermore, they could help to reduce or completely eliminate synthetic and chemical substances, for example in detergents or cleaning agents, by providing a biodegradable alternative. They could also contribute to the stabilization of other ingredients, for example.

Therefore, in a first aspect, the invention relates to a peptide comprising or consisting of an amino acid sequence of from 4 to 50 amino acids, where
(a) the amino acid sequence in N- to C-terminal orientation has the following sequence

(C)ₘX₁X₂X₃(X₄)ₙX₅(C)ₒ,

where
X₁ is a positively charged amino acid,
X₂ and X₃ are uncharged amino acids,
each X₄ is independently of one another any amino acid,
X₅ is any positively charged or uncharged amino acid,
m and o are 0 or 1, where m+o=0 or 1; and
n is an integer from 0 to 46;
or
(b) the amino acid sequence has at least 80% sequence identity to one of the amino acid sequences specified in SEQ ID NOs: 16-17, or 18-22.

It is preferred that the peptide is a peptide for adhesion to plastic surfaces, for example LSEP plastic surfaces, in particular textiles made of (LSEP) plastic or with an (LSEP) plastic content, in particular polyester.

Thus, another aspect of the invention is a peptide for adhesion to plastic surfaces comprising or consisting of an amino acid sequence of from 4 to 50 amino acids, where
(a) the amino acid sequence in N- to C-terminal orientation has the following sequence

(C)ₘX₁X₂X₃(X₄)ₙX₅(C)ₒ,

where
X₁ is a positively charged amino acid,
X₂ and X₃ are uncharged amino acids,
each X₄ is independently of one another any amino acid,
X₅ is any positively charged or uncharged amino acid,
m and o are 0 or 1, where m+o=0 or 1; and
n is an integer from 0 to 46;
or
(b) the amino acid sequence has at least 80% sequence identity to one of the amino acid sequences specified in SEQ ID NOs: 16-17, or 18-22.

In another aspect, the invention relates to a peptide for adhesion to textiles of plastic or having a plastic content, comprising or consisting of an amino acid sequence of from 4 to 50 amino acids, where
(a) the amino acid sequence in N- to C-terminal orientation has the following sequence

(C)ₘX₁X₂X₃(X₄)ₙX₅(C)ₒ,

where
X₁ is a positively charged amino acid,
X₂ and X₃ are uncharged amino acids,
each X₄ is independently of one another any amino acid,
X₅ is any positively charged or uncharged amino acid,
m and o are 0 or 1, where m+o=0 or 1; and
n is an integer from 0 to 46;
or
(b) the amino acid sequence has at least 80% sequence identity to one of the amino acid sequences specified in SEQ ID NOs: 16-17, or 18-22.

In preferred embodiments, the peptide according to the invention is functionally modified, for example, the peptide according to the invention is coupled to certain other molecules or chemical groups, e.g., organic (macro)molecules. Such molecules are also referred to as derivatives or conjugates below. In these, the peptide according to the invention acts as a type of adhesion tag that mediates binding to the desired surface or material.

In a further aspect, the invention relates to a multimer of the above peptides comprising two or more of said amino acid sequences, preferably in the form of a peptide or polypeptide.

In another aspect, the invention relates to a means comprising at least one peptide or polypeptide as described above. Also comprised are means comprising at least one peptide comprising or consisting of an amino acid sequence having at least 80%, preferably at least 90%, sequence identity to one of the amino acid sequences specified in SEQ ID NOs: 1-27, preferably 1-15, 16-17, or 18-22.

In preferred embodiments, the means is a detergent or cleaning agent, preferably a detergent.

Thus, in a further aspect, the invention relates to a detergent or cleaning agent, in particular a detergent, comprising at least one of the peptides or polypeptides described herein. In various embodiments, the peptide may comprise or consist of an amino acid sequence having at least 80%, preferably at least 90%, sequence identity to one of the amino acid sequences specified in SEQ ID NOs: 1-27, preferably 1-15, 16-17, or 18-22.

In another aspect, the invention relates to the use of the peptides or polypeptides described herein for adhesion to plastic surfaces, preferably LSEP surfaces. In such embodiments, the peptides or polypeptides may be conjugated to another molecule to be brought near the surface.

Finally, the invention also relates to the use of the peptides or polypeptides described herein for cleaning and/or treating textiles that are made of plastic or consist to some extent of plastic. Also in such embodiments, the peptides or polypeptides may be conjugated to another molecule to be brought near the surface.

In preferred embodiments, the peptide is functionally modified, e.g., derivatized or conjugated, in the agents or uses of the invention as described herein.

In preferred embodiments, the application of the peptide according to the invention or the detergent or cleaning agent according to the invention results in an improved wearing comfort of the textiles, wherein the improved wearing comfort refers, for example, to a more pleasant feel of the textile, an increased soil-repellent effect of the textile or an altered water absorption or moisture regulation.

These and other aspects, features and advantages of the invention will become apparent to a person skilled in the art through the study of the following detailed description and claims. Any feature from one aspect of the invention can be used in any other aspect of the invention. For example, features described for the peptide according to the invention are also to be used for the detergent or cleaning agent or the use according to the invention, and vice versa. Furthermore, it will readily be understood that the examples contained herein are intended to describe and illustrate but not to limit the invention and that, in particular, the invention is not limited to these examples.

Unless otherwise stated, all percentages are % by weight (wt %), based in each case on the total weight of the corresponding composition or agent. Numeric ranges specified in the format “from x to y” include the specified values. If several preferred numerical ranges are specified in this format, it is readily understood that any ranges resulting from the combination of the various endpoints are also included.

“At least one”, as used herein, refers to 1 or more, for example, 1, 2, 3, 4, 5, 6, 7, 8, 9, or more. In relation to an ingredient, it refers to the type of ingredient and not to the absolute number of molecules. “At least one peptide” thus means, for example, at least one type of peptide, i.e., that one type of peptide or a mixture of several different peptides may be meant. Together with weight information, the information refers to all compounds of the specified type contained in, for example, a composition or an agent, i.e., that the composition or agent typically does not contain any further compounds of this type beyond the specified amount of the corresponding compounds.

Numerical values specified herein without decimal places refer in each case to the full specified value with one decimal place. For example, “99%” stands for “99.0%”.

The invention relates to a peptide comprising or consisting of an amino acid sequence from 4 to 50 amino acids in length, preferably at least 8, 9, 10, 11, or 12 amino acids in length. Preferred lengths are up to 40, up to 35, up to 30, or up to 25, or up to 24 amino acids. For example, the peptide may have a length of 11, 12, 13, 14, 15, 16, 17, 18, 19, 20, 21, 22, 23, or 24 amino acids, in particular 12 to 18 amino acids.

The peptides of the invention have
(a) in N- to C-terminal orientation the following amino acid sequence

(C)ₘX₁X₂X₃(X₄)ₙX₅(C)ₒ,

where
X₁ is a positively charged amino acid, preferably R or K, more preferably R,
X₂ and X₃ are uncharged amino acids, preferably selected from A, L, M, S, I and Q, more preferably from A, S, I and L, in particular from A and L,
each X₄ is independently of one another any amino acid, preferably with the exception of P, more preferably with the exception of P and G;
X₅ is any positively charged or uncharged amino acid, preferably R or an uncharged amino acid, more preferably Q, A or L, in particular A or L,
m and o are 0 or 1, where m+o=0 or 1; and
n is an integer from 0 to 46, preferably from 6 to 20, for example 9, 10, 11, 12 or 13.

Alternatively or additionally, the peptides comprise or consist of an amino acid sequence having at least 80%, preferably at least 81%, at least 82%, at least 83%, at least 84%, or at least 85%, more preferably at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to one of the amino acid sequences specified in SEQ ID NOs: 16-17 and/or 18-22.

By a “peptide” in the context of the present invention is meant a polymer composed of amino acids, preferably the 20 proteinogenic L-amino acids, preferably of linear structure, which has up to 100 amino acids linked together by peptide bonds. According to the invention, the peptides of the invention have an amino acid sequence of 4 to 50 amino acids. In the context of the present invention, amino acids are indicated in single-letter code, where, for example, C is cysteine, R is arginine, A is alanine, and L is leucine. In particular, “C” in the above sequence (C)ₘX₁X₂X₃(X₄)ₙX₅(C)ₒ represents a cysteine residue. It is further understood that unless otherwise indicated, the amino acids in an amino acid sequence disclosed herein are linked via peptide bonds and the sequence is set forth in N- to C-terminal orientation unless otherwise indicated.

In various embodiments, the peptides of the invention may have been chemically synthesized and/or recombinantly produced by means of protein design. Nowadays, short peptides can be easily synthesized, for example via solid-phase synthesis. In contrast, longer peptides and polypeptides are often also produced recombinantly in host organisms.

In the context of the present invention, the term “N-terminus” or “N-terminal” typically describes the end of the amino acid chain of the peptide according to the invention that has a free amino group.

In the context of the present invention, the term “C-terminus” or “C-terminal” typically describes the end of the amino acid chain of the peptide according to the invention that has a free carboxyl group.

In the context of the present invention, the term “in N- to C-terminal orientation” refers to an amino acid sequence in which the sequence of amino acids are described starting from the N-terminus towards the C-terminus.

Typical acidic or negatively charged amino acids (depending on pH) are D and E.

Positively charged or basic amino acids (depending on pH) typically include R, K, and H.

Amino acids such as G, A, C, I, L, M, F, V, P, S, T, W, Y, N and Q are typically uncharged, i.e., neutral amino acids.

When reference is made herein to “any” amino acid, this usually means one of the 20 naturally occurring proteinogenic amino acids, i.e., one of glycine (G), alanine (A), valine (V), leucine (L), isoleucine (I), phenylalanine (F), serine (S), threonine (T), proline (P), methionine (M), cysteine (C), histidine (H), lysine (K), arginine (R), glutamine (Q), asparagine (N), aspartic acid (D), glutamic acid (E), tyrosine (Y) and tryptophan (W). The amino acids are typically L-amino acids unless otherwise specified. In alternative embodiments, the peptide may also consist of D-amino acids, although it may be preferred that, within the peptides described herein, D- and L-amino acids are not simultaneously present. In various embodiments, any such amino acid encompasses all of the aforementioned amino acids with the exception of proline, or in some embodiments, also with the exception of proline and glycine. In certain embodiments, these two amino acids are not preferred since they have helix-breaking properties and may therefore adversely affect the secondary structure of the peptides.

In preferred embodiments, the peptide according to the invention has a total charge of from −2 to +12, preferably from 0 to +8, more preferably from 0 to +4, in particular from 0 to +2. The total charge of the peptide is based on the number of positively and negatively charged amino acids in the peptide, in particular arginine (R), lysine (K), histidine (H), aspartic acid (D), and glutamic acid (E), and is the sum of the negative and positive charges, wherein one positive and one negative charge cancel each other out. Thus, a peptide with 2 arginine residues and 1 glutamic acid residue would have a total charge of +1. The total charge of the peptide is preferably −2 to +12, more preferably 0 to +8, even more preferably 0 to +4, in particular 0 to +2.

In preferred embodiments, the peptide according to the invention has
(i) a total charge of from −2 to +12, preferably from 0 to +8, more preferably from 0 to +4, in particular from 0 to +2; and/or
(ii) at the N-terminus comprising the first 3-4 amino acids, a net positive charge; and/or
(iii) at the C-terminus comprising the last 3-6 amino acids, a net negative or neutral charge, preferably at least one negatively charged amino acid, in particular E; and/or
(iv) no P and even more preferably also no G and in particular also no Y.

All aforementioned features, in particular (i)-(iv), may be realized individually or in any combination.

The feature that the peptide at the “N-terminus comprising the first 3-4 amino acids has a net positive charge” means that the N-terminal 3-4 amino acids comprise more positively charged amino acids than negatively charged amino acids. In various embodiments, this feature is fulfilled, for example, when the N-terminal 3-4 amino acids have 1 or 2 positively charged amino acids, i.e., H, K or R, preferably K or R, more preferably R, and no negatively charged amino acids, such as E or D. If the N-terminus contains a negatively charged amino acid, the number of positively charged amino acids must be at least 2 in order for the net charge to remain positive.

For the feature that the peptide at the “C-terminus comprising the last 3-6 amino acids has a net negative or neutral charge” means that the number of charged amino acids must be 0 or the number of negatively amino acids, i.e., D and E, must be greater than the number of positively charged ones. An example of such a C-terminal sequence would be, for example, EAL or the double sequence of this motif.

In various preferred embodiments, the sequence is X₁X₂X₃ RAL, RSI, or RLA, preferably RAL or RLA, in particular RAL. The N-terminal sequence RAL or RLA not only advantageously has a net positive charge, it also comprises amino acids with a particularly high alpha-helix-forming potential, as will be explained further below. In some embodiments, the arginine residue may also be replaced by lysine, but the N-terminal arginine residue is particularly preferred.

Furthermore, it is preferred that
(i) the sequence (X₄)ₙX₅ comprises at least one sequence X₆X₇X₈, where X₆ is a charged or uncharged amino acid, preferably R, K, E, L, A or Q, more preferably R, K, E or Q, and X₇ and X₈ are independently from one another negatively charged or uncharged amino acids with the exception of P and G, preferably A, L, E, R, Q or M, for example A, L, E, Q or M, even more preferably A, E, Q or L, even more preferably A, E or L, in particular A or L; and/or
(ii) (X₄)ₙ comprises at least one aromatic amino acid, preferably W or F.

If the sequence X₆X₇X₈ comprises an X₆ which is R or K, the sequence X₆X₇X₈ is preferably not at the C-terminus and preferably not within the 6 C-terminal amino acids. In such cases, the sequence (X₄)ₙX₅ may comprise one or more further sequences X₆X₇X₈, which are C-terminal to the sequence, which comprise X₆ a positively charged amino acid, wherein these further sequences then preferably do not have a positively charged amino acid as X₆.

It is preferred that one of the sequences X₆X₇X₈, which are close to, i.e., within the 6 C-terminal amino acids, or at the C-terminus, have as X₆ a negatively charged amino acid, for example E.

If, in some embodiments, the peptide contains an aromatic amino acid selected from W and F, a positively or negatively charged amino acid is adjacent thereto, in particular C-terminally, and in particular no other aromatic amino acid is adjacent to the aromatic amino acid.

The aromatic amino acids phenylalanine (F) and tryptophan (W) are preferably used as helix formers and/or for pi-stacking in the peptide sequence according to the invention. In various embodiments the aromatic amino acid tyrosine (Y) is not used in the peptide sequence since it has helix-breaking properties. In various embodiments, the peptide is therefore free of Y residues.

In the context of the present invention, “Pi-stacking” refers to the non-covalent interaction between aromatic ring systems.

Preferably
(i) (X₄)ₙ comprises at least one sequence X₆X₇X₈, where X₆X₇X₈ is RAL or RLA, preferably RAL, and where said sequence is preferably localized in the N-terminal amino acids of positions 4-7 or at least 6-7 amino acids away from the C-terminus; and/or
(ii) (X₄)ₙX₅ comprises at least one sequence X₆X₇X₈, where X₆X₇X₈ is EAL, LEA or ELA, preferably EAL, and where said sequence is preferably not localized in the N-terminal amino acids of positions 1-6; and/or
(iii) (X₄)ₙX₅ comprises at least one sequence X₆X₇X₈, where X₆X₇X₈ is EQA, QAL, LQA or QLA, preferably EQA, QAL or QLA, in particular QAL.

In other possible embodiments, (X₄)ₙX₅ comprises at least one sequence X₆X₇X₈, where X₆X₇X₈ is QLA or EQA, where said sequence is preferably not localized in the N-terminal amino acids of positions 1-6 or 1-11.

It is further possible that (X₄)ₙX₅ comprises at least one sequence X₆X₇X₈X₉ where X₆X₇X₈X₉ is AQLA or SEQA, where said sequence is preferably not localized in the N-terminal amino acids of positions 1-6 or 1-11.

In preferred embodiments, the peptide comprises the sequence X₁X₂X₃, where X₁X₂X₃ is RAL, and (X₄)ₙX₅ comprises at least one of QAL and EAL, preferably both. In such embodiments, the peptide has the sequence

(C)ₘRAL(X₁₀)_{q}QAL(X₁₁)ᵣEAL(X₁₂)ₛ(C)ₒ,
or
(C)ₘRAL(X₁₀)_{q}EAL(X₁₁)ᵣQAL(X₁₂)ₛ(C)ₒ,

where X₁₀ and X₁₁ independently of one another are any amino acid, preferably with the exception of P, more preferably with the exception of P and G;
X₁₂ is any uncharged amino acid, preferably Q, A or L, in particular A or L;
q and r are 0 or integers of 1-10, preferably 0, 1, 2 or 3; and
s is 0 or 1,
where q+r+s=0-21, preferably 0-15 or 0-9 or 1-15 or 1-9 or 1-6 or 0-6 or 0-3 or 1-3.

Furthermore, it is preferred that the peptide additionally comprises at least one further (second) sequence RAL. Said sequence may, in various embodiments, directly C-terminally follow the first RAL sequence or be separated from it by 1-3 amino acids, for example by 1 or 3 three amino acids.

In preferred embodiments, the peptide contains two sequences RAL and at least one sequence each of EAL and QAL. Preferred sequences are:

RALRAL(X₁₀)_{q}QAL(X₁₁)ᵣEAL(X₁₂)ₛ,
RALRAL(X₁₀)_{q}EAL(X₁₁)ᵣQAL(X₁₂)ₛ
RAL(X₁₀)_{q}RALQAL(X₁₁)ᵣEAL(X₁₂)ₛ,
RAL(X₁₀)_{q}RALEAL(X₁₁)ᵣQAL(X₁₂)ₛ
RAL(X₁₀)_{q}QALRAL(X₁₁)ᵣEAL(X₁₂)ₛ,
RAL(X₁₀)_{q}EALRAL(X₁₁)ᵣQAL(X₁₂)ₛ,

where X₁₀ and X₁₁ independently of one another are any amino acid, preferably with the exception of P, more preferably with the exception of P and G, for example W or F, or comprises further motifs QAL or EAL;
X₁₂ is any uncharged amino acid, preferably Q, A or L, in particular A or L;
q and r are 0 or integers of 1-6, preferably 0, 1, 2 or 3; and
s is 0 or 1, preferably 0,
where q+r=0-9, preferably 0-6 or 0-3 or 1-9 or 1-6 or 1-3.

Furthermore, it is preferred in various embodiments that the peptide contains at least one W or F, preferably exactly one W or F.

Preferably, the peptide according to the invention comprises amino acids having a high alpha-helix-forming potential, with said amino acids being selected from E, A, L, M, Q, K, R, F, I, H, W and D, more preferably E, A, L, M, Q, K, R, F, I and H; even more preferably E, A, L, M, Q, K, R and F.

In preferred embodiments, the peptide consists of at least 60%, preferably at least 65%, more preferably at least 70%, in particular at least 75% or at least 80% or at least 85% or at least 90% or at least 95% of amino acids having a high alpha-helix-forming potential, with said amino acids being preferably selected from E, A, L, M, Q, K, R, F, I, H, W and D, more preferably E, A, L, M, Q, K, R, F, I and H; even more preferably E, A, L, M, Q, K, R and F.

Particularly preferably, the peptide according to the invention forms a helical secondary structure, in particular an α-helical structure, preferably with an α-helical content of at least 80%, preferably at least 85%, more preferably at least 90%, even more preferably at least 95%, in particular higher than 95%. The use of the AL or LA motif in the amino acid sequence of the peptide according to the invention can contribute to the stability of the helix structure because these amino acids have a high α-helix potential.

In preferred embodiments, the peptide according to the invention has an amino acid sequence according to one of SEQ ID NOs: 1-15 and/or 16-17, in particular 1-15, or variants thereof, which has at least 80%, preferably at least 81%, at least 82%, at least 83%, at least 84% or at least 85%, more preferably at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% sequence identity to the specified sequence, wherein preferably the RAL motif, and more preferably also the EAL and/or QAL motif, if present, are invariant. If the RAL, EAL and QAL motifs are present in the peptide, they are preferably invariable in all the aforementioned variants.

In various embodiments, the peptide may have a high proportion of hydrophobic amino acids selected from A, L, F, W, V, M, I, and P, in particular A, L, F, W, V, M, and I.

Preferably, the peptide has an amino acid sequence which has a length of 10 to 24 amino acids, for example 11, 12, 13, 14, 15, 16, 17, 18, 19, 20, 21, 22, 23 or 24 amino acids, in particular 12 to 19 amino acids, for example 12-18 amino acids.

In some embodiments, the peptide according to the invention has the amino acid cysteine (C) at the C-terminus. In other embodiments, the peptide according to the invention has the amino acid cysteine at the N-terminus. This amino acid can enable coupling to other molecules, structures or substrates via the free sulfhydryl group. This amino acid therefore serves as a linkage point but is typically not involved in the desired adhesive effect.

Preferably, the peptide according to the invention comprises or consists of an amino acid sequence which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5%, in particular 100% sequence identity to one of the following amino acid sequences:

(SEQ ID NO: 1)
RALQALRALQALEAL,
(SEQ ID NO: 2)
RALRALRALEALEAL,
(SEQ ID NO: 3)
RALRALRALQALQAL,
(SEQ ID NO: 4)
RALRALRALQALEAL
(SEQ ID NO: 5)
RALRALQALEALEAL,
(SEQ ID NO: 6)
RALFEALQALFRALEAL,
(SEQ ID NO: 7)
RALRALEALQALEA,
(SEQ ID NO: 8)
RALFEALFRALEALR,
(SEQ ID NO: 9)
RALFEALFRALEAL,
(SEQ ID NO: 10)
RALEALFRALEAL,
(SEQ ID NO: 11)
RALRALFEALEAL,
(SEQ ID NO: 12)
RALEALFRALQALEAL,
(SEQ ID NO: 13)
RALEALWRALQALEAL,
(SEQ ID NO: 14)
RALEALWRALEAL,
(SEQ ID NO: 15)
RALARALARALAQALA,
(SEQ ID NO: 16)
RSIVTFSLRQNAQLA
or
(SEQ ID NO: 17)
RSIVTFSLRQNSEQA,
preferably 1-15.

In various other embodiments, the peptide according to the invention comprises or consists of an amino acid sequence which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5%, in particular 100% sequence identity to one of the following amino acid sequences: RSIVTFSLRQNAQLA (SEQ ID NO: 16), RSIVTFSLRQNSEQA (SEQ ID NO: 17), GLHTSATNLYLH (SEQ ID NO: 18), QHSIRLLTIKKP (SEQ ID NO: 19), QQSIRIMTIKHP (SEQ ID NO: 20), WRHPRLRCGNLL (SEQ ID NO:21) or QKSRNRMTRTHP (SEQ ID NO: 22), preferably 16 or 17.

In various other embodiments, the peptide according to the invention comprises or consists of an amino acid sequence which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5%, in particular 100% sequence identity to one of the following amino acid sequences:

(SEQ ID NO: 18)
GLHTSATNLYLH,
(SEQ ID NO: 19)
QHSIRLLTIKKP,
(SEQ ID NO: 20)
QQSIRIMTIKHP,
(SEQ ID NO: 21)
WRHPRLRCGNLL
or
(SEQ ID NO: 22)
QKSRNRMTRTHP

In various embodiments, the peptide according to the invention may further comprise or consist of one of the following amino acid sequences, or a variant thereof: SRARLFVVTYHK (SEQ ID NO: 23), HMISTMNAASRR (SEQ ID NO: 24), RSIVTFSLRQNR (SEQ ID NO: 25), RNTIRIRTIKHP (SEQ ID NO: 26), or RHSSTLRYRPLP (SEQ ID NO: 27), wherein a variant has an amino acid sequence which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5% sequence identity to one of the amino acid sequences.

Variants of the peptides according to the invention, the amino acid sequence of which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5% sequence identity to one of the amino acid sequences which correspond to the sequences in SEQ ID NOs: 1-27, preferably 1-22, more preferably 1-15, 16-17 or 18-22, more preferably 1-15 or 16-17, in particular 1-15, preferably differ in a maximum of 3 positions, more preferably in a maximum of 2 positions, in particular in a maximum of 1 position from one of the amino acid sequences, which correspond to the sequences in SEQ ID NOs: 1-27, preferably 1-22, more preferably 1-15, 16-17, or 18-22, more preferably 1-15 or 16-17, in particular 1-15. For example, if 1 position is different, either one of the amino acids of the respective sequence can be replaced or else the sequences match, but an amino acid within the amino acid chain or N- or C-terminal has been added or omitted. For example, a cysteine (C) could have been attached to the C- or N-terminal.

The term “variant”, as used herein, refers to variants of the peptide according to the invention which continue to have the functionality of the starting peptide but differ from the starting sequence by one or more sequence deviations, for example 1, 2 or 3 sequence deviations, for example a substitution, deletion or insertion. The sequence identity of such variants may be in the range of 80% based on the total length of the starting peptide, and may be at least 81, 82, 83, 84, 85, 86, 87, 88, 89, 90, 91, 92, 93, 94, 95, 96, 97, 98, 99 or 99.5%.

The identity of nucleic acid or amino acid sequences is determined by a sequence comparison. This sequence comparison is based on the BLAST algorithm, which is established and commonly used in the prior art (cf., for example, Altschul, S. F., Gish, W., Miller, W., Myers, E. W. & Lipman, D. J. (1990) “Basic local alignment search tool.” J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs”; Nucleic Acids Res., 25, S.3389-3402) and in principle occurs in that similar sequences of nucleotides or amino acids in the nucleic acid or amino acid sequences are assigned to one another. A tabular assignment of the relevant positions is referred to as an alignment. A further algorithm available in the prior art is the FASTA algorithm. Sequence comparisons (alignments), in particular multiple sequence comparisons, are created using computer programs. Frequently used are for example the Clustal series (cf., for example, Chenna et al. (2003): Multiple sequence alignment with the Clustal series of programs. Nucleic Acid Research 31, 3497-3500), T-Coffee (cf., for example, Notre dame et al. (2000): T-Coffee: A novel method for multiple sequence alignments. J. Mol. Biol. 302, 205-217) or programs based on these programs or algorithms. Also possible are sequence comparisons (alignments) using the computer program Vector NTIR Suite 10.3 (Invitrogen Corporation, 1600 Faraday Avenue, Carlsbad, California, USA) with the given standard parameters, the AlignX module of which is based on ClustalW for the sequence comparisons. Unless stated otherwise, the sequence identity indicated herein is determined using the BLAST algorithm.

Such a comparison also allows a conclusion to be drawn about the similarity of the compared sequences to one another. It is usually given in percent identity, i.e., the proportion of identical nucleotides or amino acid residues at the same positions or positions corresponding to one another in an alignment. In the case of amino acid sequences, the broader concept of homology takes conserved amino acid exchanges into account, i.e., amino acids having similar chemical activity, as these usually perform similar chemical activities within the peptide/protein. Therefore, the similarity of the compared sequences can also be indicated as percent homology or percent similarity. Identity and/or homology information can be provided regarding whole peptides, polypeptides or genes or only regarding individual regions. Homologous or identical regions of different nucleic acid or amino acid sequences are therefore defined by matches in the sequences. Such regions often have identical functions. They can be small and comprise only a few nucleotides or amino acids. Such small regions, however, often perform essential functions for the overall activity of the peptide/protein. It may therefore be expedient to relate sequence matches only to individual, optionally small, regions. Unless otherwise stated, however, identity or homology information in the present application relates to the total length of the respective nucleic acid or amino acid sequence indicated.

The peptide or protein concentration can be determined using known methods, for example the BCA method (bicinchoninic acid; 2,2′-bichinolyl-4,4′-dicarboxylic acid) or the Biuret method (A. G. Gornall C. S. Bardawill and M. M. David, J. Biol. Chem., 177 (1948), pp. 751-766). A person skilled in the art in the field of peptide and protein technology is aware of a variety of suitable methods for determining peptide or protein concentration that can be used within the scope of this invention.

Peptides according to the invention may have amino acid changes, in particular amino acid substitutions, insertions or deletions. Such peptides are, for example, further developed by targeted genetic alteration, i.e., by mutagenesis methods, and optimized for specific purposes or with regard to specific properties (for example with regard to their stability, binding etc.).

For example, targeted mutations such as substitutions, insertions or deletions can be introduced into the known molecules in order to alter certain properties, for example. For this purpose, in particular the surface charges and/or the isoelectric point of the molecules and thus their interactions with a surface can be altered. For example, the net charge of the peptides can be altered in order to thereby influence the substrate binding. Alternatively or additionally, one or more corresponding mutations can, for example, increase the stability or adsorption of the peptide. Advantageous properties of individual mutations, e.g., individual substitutions, can complement one another.

Thus, the invention also comprises peptides characterized in that they are obtainable from a peptide as described above as starting molecule, for example from a molecule having one of the amino acid sequences according to SEQ ID NOs: 1-27, preferably 1-22, more preferably 1-15, 16-17 and/or 18-22, even more preferably 1-15 or 16-17, in particular 1-15, on which, for example, one or more amino acid substitutions, including, inter alia, single or multiple conservative amino acid substitutions, have been carried out, wherein the resulting peptide has at least 80% sequence identity to one of the amino acid sequences according to SEQ ID NOs: 1-27, preferably 1-22, more preferably 1-15, 16-17 or 18-22, even more preferably 1-15 or 16-17, in particular 1-15.

The term “conservative amino acid substitution” refers to the exchange (substitution) of one amino acid residue for another amino acid residue, wherein this exchange does not result in a change in polarity or charge at the position of the exchanged amino acid, such as the exchange of a non-polar amino acid residue for another non-polar amino acid residue. Conservative amino acid substitutions within the scope of the invention comprise, for example: G=A=S, I=V=L=M, D=E, N=Q, K=R, Y=F, S=T, G=A=I=V=L=M=Y=F=W=P=S=T. However, it may be preferred for such exchanges not to have glycine or tyrosine as the target amino acid or, for example, also not to have an amino acid which has a low alpha-helix-forming potential.

In preferred embodiments, the peptide according to the invention may also be modified. Preferred modifications can be, for example, coupling of the peptide according to the invention with particular other molecules or chemical groups, for example organic (macro)molecules, e.g., via a covalent bond or a linker via a suitable amino acid of the chain and/or N- and/or C-terminally.

If the peptide according to the invention is coupled with at least one further (macro)molecule, it may also be referred to as a peptide derivative. The peptide according to the invention is then derivatized. In such embodiments, the peptide can act as an adhesion tag, which causes a molecule coupled thereto to bind to the desired surface. Such molecules may also be referred to as conjugates.

In some embodiments, the mentioned peptides according to the invention which may, for example for coupling purposes, N- and/or C-terminally have the amino acid cysteine, are coupled (functionally modified) with biotin, preferably at a suitable amino acid of the chain and/or N- and/or C-terminally.

It is also possible that the peptide is part of a protein or polypeptide. Such proteins and polypeptides can be produced recombinantly, for example, as fusion proteins. In such embodiments, the peptide according to the invention is either N- or C-terminal localized to provide the adhesion of the whole molecule to a surface. In such embodiments, too, the peptide thus acts as adhesion tag.

In various preferred embodiments, the peptide according to the invention may
(i) comprise or consist of any of the following amino acid sequences:

(SEQ ID NO: 28)
RALQALRALQALEAL-C,
(SEQ ID NO: 29)
RALRALRALEALEAL-C,
(SEQ ID NO: 30)
RALRALRALQALQAL-C,
(SEQ ID NO: 31)
RALRALRALQALEAL-C,
(SEQ ID NO: 32)
RALRALQALEALEAL-C,
(SEQ ID NO: 33)
RALFEALQALFRALEAL-C,
(SEQ ID NO: 34)
RALRALEALQALEA-C,
(SEQ ID NO: 35)
RALFEALFRALEALR-C,
(SEQ ID NO: 36)
RALFEALFRALEAL-C,
(SEQ ID NO: 37)
RALEALFRALEAL-C,
(SEQ ID NO: 38)
RALRALFEALEAL-C,
(SEQ ID NO: 39)
RALEALFRALQALEAL-C,
(SEQ ID NO: 40)
RALEALWRALQALEAL-C,
(SEQ ID NO: 41)
RALEALWRALEAL-C,
(SEQ ID NO: 42)
RALARALARALAQALA-C,
(SEQ ID NO: 43)
RSIVTFSLRQNAQLA-C,
(SEQ ID NO: 44)
RSIVTFSLRQNSEQA-C,
in particular one of SEQ ID NOs: 28-36,
39-44, in particular 28-32, 34, 42-44;

or
(ii) comprise or consist of an amino acid sequence which has at least 80%, preferably at least 81%, 82%, 83%, 84% or 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5% sequence identity to one of the sequences specified in SEQ ID NOs: 28-44 (28, 29, 30, 31, 32, 33, 34, 35, 36, 37, 38, 39, 40, 41, 42, 43 or 44), preferably with one of SEQ ID NOs: 28-36, 39-44, in particular 28-32, 34, 42-44.

Furthermore, it is possible for the peptide according to the invention to
(i) comprises or consists of one of the following amino acid sequences:

(SEQ ID NO: 45)
GLHTSATNLYLH-C,
(SEQ ID NO: 46)
QHSIRLLTIKKP-C,
(SEQ ID NO: 47)
QQSIRIMTIKHP-C,
(SEQ ID NO: 48)
WRHPRLRCGNLL-C,
or
(SEQ ID NO: 49)
QKSRNRMTRTHP-C;

or
(ii) comprises or consists of an amino acid sequence which has least 80%, preferably at least 81%, 82%, 83%, 84% or 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5% sequence identity to one of the sequences specified in SEQ ID NOs: 45-49 (45, 46, 47, 48 or 49).

In various embodiments, the peptide may further comprise or consist of an amino acid sequence according to SEQ ID NOs: 50-54 (SRARLFVVTYHK-C(SEQ ID NO: 50), HMISTMNAASRR-C(SEQ ID NO: 51), RSIVTFSLRQNR-C(SEQ ID NO: 52), RNTIRIRTIKHP-C-(SEQ ID NO: 53), RHSSTLRYRPLP-C(SEQ ID NO: 54)) or a variant thereof, which has at least 80%, preferably at least 81%, 82%, 83%, 84% or 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5% sequence identity therewith.

In certain embodiments, the peptide is a peptide suitable for adhesion to plastic surfaces.

In the context of this invention, “adhesion” is understood to mean an interaction between the peptide and a surface, thereby allowing the peptide to adhere to the surface. Thus, an “adhesive peptide” refers to a peptide that has the ability to interact with a particular surface and/or to adhere to a particular surface. The adhesive peptides described herein preferably have a 10-fold, more preferably a 20-fold, 50-fold or 100-fold greater adhesion to a given surface than any alternative peptide of comparable length which has not been developed for this purpose and does not meet the sequence specifications described herein.

The terms “synthetic” and “plastic” are to be used synonymously and typically refer to solids comprising polymers as basic building blocks, although other (chemical) groups and molecules may be added, for example.

The term “solid” in this context refers to any property of solid substances, i.e., it is to be distinguished from liquid or gaseous substances. Solids can be hard and inflexible, as well as ductile, bendable and flexible.

It is particularly preferred that the plastic surface is a plastic surface with low surface energy (low surface energy polymer (LSEP) surface). These surfaces are typically inert surfaces which had to be activated first for functionalization or for further modifications. Common methods here include, but are not limited to, plasma or corona treatment as well as treatment with primers, such as solvents or oxidizing agents. Such pretreatment methods can be circumvented or avoided by utilizing the peptides described herein because the peptides described also bind the untreated LSEP surfaces well.

To determine the surface energy of a plastic surface, a contact angle measurement can be carried out, for example, without being limited thereto. The measured contact angle indicates to what extent a liquid can wet the surface of a solid. It can be read from this to what extent this liquid, for example an agent according to the invention, is suitable for treating the surface being examined. For a person skilled in the art in this field, contact angle measurement is a common measurement method.

Preferably, the plastic surface comprises or consists of a material selected from the following: polyester (PES), polyethylene (PE), polypropylene (PP), polyurethane (PU), polystyrene (PS), polyvinyl chloride (PVC), polycarbonate (PC), polyamide (PA), polyphenylene ether, polyphenylene sulfide, polyoxymethylene (POM), polymethyl methacrylate (PMA), polyethylene terephthalate (PET), polybutylene terepthalate (PBT), polytetrafluoroethylene (PTFE), polyhydroxyalkanoate (PHA), polyhydroxybutyrate (PHB) polyimide (PI), polylactide (PLA), polyvinylidene fluoride (PVDF), polyether ketone (PEK etc.), copolymers and/or mixtures thereof and/or polymeric foams thereof, preferably polyester (PES), polyethylene (PE), polypropylene (PP), polystyrene (PS), copolymers and/or mixtures thereof and/or polymeric foams thereof.

In various embodiments, the plastic surface material is polyethylene (PE), polypropylene (PP), polystyrene (PS), copolymers and/or mixtures thereof and/or polymeric foams thereof.

Preferably, the plastic surface may represent one or more of the following parts: composite components, films, packaging, plastic particles, and/or textiles, for example functional textiles.

Preferably, the peptide according to the invention demonstrates adsorption to the plastic surface of 0.25 to 3, for example 0.25 to 1 or 0.4 to 1, measured by means of the semi-quantitative method for adhesion measurement, which is described, for example, in WO 2014/072313 A1.

Using the direct BCA method for adhesion measurement, the preferred adhesion values are in the range of 25 to 100, in particular 40 to 100. This method is described, for example, in Example 2.

Good adhesion to plastic surfaces can be achieved, for example, by a peptide having a pronounced helix structure (in particular α-helix structure) and/or a high arginine content, in particular at one of the termini.

Peptides according to the invention, for adhesion to plastic surfaces, preferably to LSEP surfaces, in particular to textiles made of (LSEP) plastic or with an (LSEP) plastic content, comprise or consist of an amino acid sequence of 4 to 50 amino acids, preferably 10 to 24 amino acids, for example 11, 12, 13, 14, 15, 16, 17, 18, 19, 20, 21, 22, 23 or 24 amino acids, in particular 12 to 19 amino acids, for example 12 to 18, where
(a) the amino acid sequence in N- to C-terminal orientation has the following sequence

(C)ₘX₁X₂X₃(X₄)ₙX₅(C)ₒ,

X₁ is a positively charged amino acid, preferably R or K, more preferably R,
X₂ and X₃ are uncharged amino acids, preferably selected from A, L, M, S, I and Q, more preferably from A, L, S and I or A, L, M and Q, in particular from A and L,
each X₄ is independently of one another any amino acid, preferably with the exception of P, more preferably with the exception of P and G;
X₅ is any positively charged or uncharged amino acid, preferably R or an uncharged amino acid, more preferably Q, A or L, in particular A or L,
m and o are 0 or 1, where m+o=0 or 1; and
n is an integer from 0 to 46, preferably from 6 to 20, for example 9, 10, 11, 12 or 13;
or
(b) the amino acid sequence has at least 80%, preferably at least 85%, more preferably at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or 100% sequence identity to one of the amino acid sequences specified in SEQ ID NOs: 16-22, preferably 16-17 or 18-22.

Particularly preferred peptides according to the invention, which exhibit good adhesion in particular to polyester, have an amino acid sequence which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5%, in particular 100% sequence identity to one of the amino acid sequences corresponding to the sequences in SEQ ID NOs. 1-17 or 28-44, in particular 1, 2, 5-9 and 12-17 or 28, 29, 32-36 and 39-44, in particular 2, 5, 7, 16 and 17 or 29, 32, 34, 43 and 44.

In some embodiments, a peptide that exhibits good adhesion, in particular to polyester, may have an amino acid sequence that has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5%, in particular 100% sequence identity to one of the amino acid sequences corresponding to the sequences in SEQ ID NOs. 18-22 and/or 23-27 or 45-49 and/or 50-54, preferably 19, 21-23, 25 and/or 27 or 46, 48-50, 52 and/or 54, in particular 23 and 25 or 50 and 52.

Methods for determining the adhesion of the peptides according to the invention are described in the examples, although the determination is not limited to these methods and the person skilled in the art in this field can apply all known and suitable methods for determining adhesion.

Furthermore, the peptide according to the invention may also be at least one subunit (module) of a peptide or polypeptide, wherein the polypeptide may comprise a multimer of the sequences described herein, for example 1 to 30 repeats, more preferably 2 to 15 repeats, in particular 2 to 10 repeats, for example 2, 3, 4, 5 or 6 repeats of the peptides according to the invention. The polypeptide may comprise or consist of such multimers. In various embodiments, the individual peptide subunits of the polypeptide (multimer) can be separated from one another by spacers, for example by spacers having a length of 1 to 10 amino acids, preferably 1 to 4 amino acids.

Thus, a further aspect of the invention is also a peptide or polypeptide (multimer) comprising one or more, preferably two or more, peptides according to the invention, as described herein. In this context, the term “polypeptide” refers in particular to such peptides comprising 100 or more amino acids.

The peptides according to the invention can be chemically produced by known methods of peptide synthesis, for example by solid phase synthesis according to Merrifield.

However, it is preferred to produce the peptide according to the invention using recombinant methods. According to the invention, these are understood to mean all genetic engineering or microbiological methods which are based on introducing the genes for the peptides of interest into a host organism that is suitable for production, and the host organism transcribing and translating the genes (amalgamated in the context of this invention as biotechnological methods). For example, the introduction of the genes of interest occurs via vectors, in particular expression vectors; but also via such vectors that cause the gene of interest to be able to be inserted into an existing genetic element in the host organism, such as the chromosome or other vectors. The functional unit composed of a gene and a promoter and possibly other genetic elements is typically referred to as an “expression cassette.” However, for this purpose the expression cassette does not necessarily also have to be present as a physical unit.

Particularly preferably, the peptides according to the invention are produced as polypeptides (multimers) and subsequently cleaved into the functional peptides. Very particularly preferred multimers have 2 to 10 peptide units (each according to the invention), which in each case are separated from one another by spacers having a length of 0 to 4 amino acids (for example 1, 2, 3 or 4 amino acids). These spacers can consist, for example, of the amino acids glycine (G), alanine (A) and serine (S). Alternatively, the spacers may be or comprise interfaces for specific proteases/peptidases, in particular endopeptidases, or form such an interface together with parts of the peptide according to the invention.

A person skilled in the art is able, by means of nowadays generally known methods such as chemical synthesis or polymerase chain reaction (PCR) in connection with standard molecular biological and/or protein chemical methods, to produce the corresponding nucleic acids and even complete genes on the basis of known DNA and/or amino acid sequences and to then utilize these to synthesize peptides and polypeptides in suitable host cells.

In particularly preferred embodiments, the peptide is produced by means of biotechnological methods, as defined above.

In the context of the present invention, vectors are understood to mean elements which consist of nucleic acids and which contain a gene of interest as a characterizing nucleic acid range. Vectors allow establishment of this gene in a species or a cell line over multiple generations or cell divisions as a stable genetic element that is replicating itself independently of the rest of the genome. Vectors are, in particular when used in bacteria, special plasmids, i.e., circular genetic elements. In genetic engineering, a distinction is made between, on the one hand, such vectors that are used for storage, and thus, in a manner of speaking, also for genetic engineering work, the so-called cloning vectors, and, on the other hand, those vectors that fulfill the function of realizing the gene of interest in the host cell, i.e., of enabling the expression of the peptide in question. These vectors are referred to as expression vectors.

The nucleic acid encoding a peptide according to the invention or a multimer of such a peptide, which constitutes a further aspect of the invention, may be cloned into a vector, for example. The molecular biological dimension of the invention, therefore, lies in vectors having the genes for the corresponding peptides. These may include vectors, for example, which are derived from bacterial plasmids, from viruses, or from bacteriophages, or predominantly synthetic vectors or plasmids with elements from a wide variety of origins. Using the further genetic elements which are present in each case, vectors are able to become established as stable units in the host cells in question over several generations. In the context of the invention, it is immaterial whether the vectors are established extrachromosomally as independent units, or are integrated into a chromosome. Which of the numerous systems known from the prior art is selected depends on the individual case. For example, the achievable copy number, the available selection systems, among them primarily antibiotic resistance, or the culturability of the host cells that are capable of accepting the vectors may be deciding factors.

The vectors form suitable starting points for molecular biological and biochemical investigations of the gene in question or the associated peptide, for developments according to the invention, and lastly for amplifying and producing peptides according to the invention. The vectors represent further aspects of the present invention.

Cloning vectors are preferred embodiments of the present invention. In addition to storage, they are suitable for biological amplification or selection of the gene of interest for characterization of the gene in question, for example via the creation of a restriction map or via sequencing. Cloning vectors are therefore also preferred embodiments of the present invention, since they represent a transportable and storable form of the claimed DNA. They are also preferred starting points for molecular biological techniques that are not tied to cells, for example the polymerase chain reaction.

Expression vectors are chemically similar to the cloning vectors, but differ in those partial sequences which enable the expression vectors to replicate in the host organisms which are optimized for the production of peptides and to express the gene contained therein. Expression vectors, which themselves bear the genetic elements necessary for expression, are preferred embodiments. The expression is influenced, for example, by promoters which regulate the transcription of the gene. Thus, the expression may take place due to the natural promoter which is originally localized in front of this gene, but also after genetic fusion, both by a promoter of the host cell provided on the expression vector or also by a modified or completely different promoter of another organism.

Expression vectors which can be regulated via changes in the culture conditions, such as the cell density or specific factors, or by adding certain compounds, are preferred embodiments.

Embodiments of the present invention may also include cell-free expression systems in which peptide biosynthesis is replicated in vitro. Such expression systems are likewise established in the prior art.

In vivo synthesis of a peptide according to the invention, i.e. by living cells, requires transfer of the associated gene into a host cell, what is known as the transformation thereof. In principle, all organisms, i.e. prokaryotes, eukaryotes, or Cyanophyta, are suitable as host cells. Preferred host cells are those which may be easily managed genetically, which concerns, for example, transformation using the expression vector and stable establishment thereof, for example unicellular fungi, such as, for example, yeasts or bacteria. In addition, preferred host cells are characterized by good microbiological and biotechnological manageability. This relates, for example, to ease of culturing, high growth rates, low demands on fermentation media, and good production and secretion rates for foreign peptides. It is often necessary to experimentally determine the optimal expression systems for the individual case from the large number of different systems that are available according to the prior art. Each peptide according to the invention may be obtained in this way from a variety of host organisms.

Such host cells are a further aspect of the present invention. Those host cells of which the activity thereof can be regulated due to genetic regulation elements which are provided on the expression vector, for example, but which may also be present in these cells from the outset, represent preferred embodiments. Expression in said cells may be induced, for example, by controlled addition of chemical compounds used as activators, by changing the cultivation conditions or when a particular cell density is reached. This allows very cost-effective production of the peptides of interest.

Prokaryotic or bacterial cells are preferred host cells. Compared to eukaryotes, bacteria are generally characterized by shorter generation times and lower demands on the culturing conditions. Inexpensive methods for obtaining peptides according to the invention may thus be established. In the case of gram-negative bacteria, such as E. coli, a large number of peptides are secreted into the periplasmatic space, i.e., into the compartment between the two membranes enclosing the cells. This can be advantageous for specific applications. In contrast, gram-positive bacteria, such as Bacilli or Actinomycetes or other representatives of the Actinomycetales, do not have an outer membrane, so that secreted peptides are immediately released into the culture medium surrounding the cells, from which, according to another preferred embodiment, the expressed peptides according to the invention may be directly purified.

A variant of this principle is represented by expression systems in which additional genes, for example those provided on other vectors, influence the production of peptides according to the invention. These may be modified gene products, or gene products which are to be purified together with the peptide according to the invention.

On account of the extensive experience with, for example, molecular biological methods and the culturability with coliform bacteria, these represent preferred embodiments of the present invention. Particularly preferred are those of the genera Escherichia coli, in particular non-pathogenic strains suitable for biotechnological production.

Representative members of these genera are the K12 derivatives and the B strains of Escherichia coli. Strains which can be derived therefrom by genetic and/or microbiological methods known per se, and can thus be regarded as their derivatives. These are of greatest importance for genetic and microbiological work and are preferably used to develop methods according to the invention. Such derivatives may be altered with regard to their requirements for culture conditions, for example via deletion or insertion mutagenesis, may have different or additional selection markers, or may express different or additional peptides. These may in particular be those derivatives which, in addition to the peptide produced according to the invention, express further peptides of economic interest.

Preferred microorganisms are also those characterized by having been obtained after transformation using one of the vectors described above. These may be, for example, cloning vectors that have been introduced for storage and/or modification in any bacterial strain. Such steps are widespread in the storage and development of genetic elements in question. Since the relevant genetic elements can be directly transferred from these microorganisms into gram-negative bacteria suitable for expression, the previous transformation products are also realizations of the relevant subject matter of the invention.

Eukaryotic cells may also be suitable for producing peptides according to the invention. Examples thereof are fungi such as Actinomycetes or yeasts such as Saccharomyces or Kluyveromyces. This may be particularly advantageous, for example, when, in the context of their synthesis, the peptides undergo specific modifications which allow such systems. This includes, for example, the binding of low-molecular compounds such as membrane anchors or oligosaccharides. In the context of this invention, this would be an example of a functionally modified peptide.

The host cells of the method according to the invention are cultured and fermented in a manner known per se, for example in discontinuous or continuous systems. In the first case, a suitable culture medium is inoculated with the recombinant bacterial strains, and the product is harvested from the medium after a period of time that is to be experimentally determined. Continuous fermentations are characterized by reaching a steady state in which cells partially die but also regrow over a comparatively long period of time and at the same time product can be removed from the medium.

Fermentation methods are well known per se from the prior art, and represent the actual large-scale production step, followed by a suitable purification method.

All fermentation methods which are based on one of the above-mentioned methods for producing recombinant peptides represent correspondingly preferred embodiments of this subject matter of the invention.

In this regard, the conditions that are optimal in each case for the production methods used, for the host cells and/or the peptides to be produced, must be experimentally determined according to the knowledge of a person skilled in the art, based on the previously optimized culture conditions of the strains in question, for example with regard to fermentation volume, media composition, oxygen supply, or agitator speed.

Fermentation methods, which are characterized in that the fermentation is carried out via an inflow strategy, are also considered. Here, the media components consumed by the continuous cultivation are fed; this is also referred to as a feeding strategy. Significant increases both in the cell density and in the dry biomass, and/or primarily in the activity of the peptide of interest, may be achieved in this way.

Analogously, the fermentation may also be designed in such a way that undesirable metabolic products are filtered out, or neutralized by adding a buffer, or counterions which are appropriate in each case.

The produced peptide may be subsequently harvested from the fermentation medium. This fermentation method is preferred over product preparation from the dry mass, but requires the provision of suitable secretion markers and transport systems.

In the absence of secretion, it is necessary, in certain circumstances, to purify the peptide from the cell mass; various methods for this purpose are also known, such as precipitation by ammonium sulfate or ethanol, for example, or chromatographic purification, to the point of homogeneity, if necessary. However, the majority of the described technical methods should manage with an enriched, stabilized preparation.

All of the elements already described above may be combined into a method in order to produce peptides according to the invention. These methods for producing the peptides according to the invention represent further aspects of the present invention. Numerous possible combinations of method steps are conceivable for each peptide according to the invention. Optimal conditions could be determined experimentally for each specific individual case.

A further aspect of the present invention further are (isolated) nucleic acid sequences encoding a peptide according to the invention, or encoding a peptide comprising or consisting of an amino acid sequence according to one of SEQ ID NOs: 1-27, preferably 1-15 and/or 16-17 and/or 18-22, more preferably 1-15 and/or 16-17, in particular 1-15, or variants thereof which have at least 90%, preferably at least 91%, more preferably at least 92%, more preferably at least 93%, even more preferably at least 94%, even more preferably at least 95%, even more preferably at least 96%, even more preferably at least 97%, even more preferably at least 98%, even more preferably at least 99%, in particular at least 99.5% sequence identity to the sequences mentioned.

Furthermore, the invention relates to vectors, in particular cloning or expression vectors, comprising at least one (isolated) nucleic acid sequence according to the invention.

In a further aspect, the invention relates to a host cell which is transformed with and/or contains at least one (isolated) nucleic acid sequence according to the invention or a vector according to the invention.

In a further aspect, the invention relates to a method for producing the peptides according to the invention.

In the aspects mentioned, the peptide according to the invention may also be a peptide or polypeptide comprising one or more, preferably two or more, peptides according to the invention as described herein.

Furthermore, in one aspect, the invention relates to means comprising at least one peptide or polypeptide according to the invention. Said peptide may consist of or comprise an amino acid sequence having at least 80%, preferably at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or 100% sequence identity to one of the amino acid sequences specified in SEQ ID NOs: 1-27, preferably 1-15, 16-17 or 18-22, more preferably 1-15 or 16-17, in particular 1-15.

Preferably, the means comprises peptides as described herein for adhesion to plastic surfaces, preferably for adhesion to LSEP surfaces, in particular to textiles made of (LSEP) plastic or having a (LSEP) plastic content, in particular polyester, polyethylene, polypropylene or polystyrene or copolymers or mixtures thereof. In these applications, the peptide may be conjugated to another molecule.

In various embodiments, the means according to the invention is
(i) a coating agent, a varnish, a paint, an adhesion promoter or an adhesive, preferably a water-based adhesive; and/or
(ii) a detergent or cleaning agent.

The pH of the agent according to the invention is preferably 2 to 12, preferably 5 to 9, more preferably 7 to 9, both in concentrated form and in diluted application solution.

Particularly preferably, the means according to the invention is a detergent or cleaning agent, in particular a detergent.

Thus, detergent or cleaning agents comprising at least one peptide as described herein are also an aspect of the invention, in particular those comprising or consisting of an amino acid sequence having at least 80%, preferably at least 81%, at least 82%, at least 83%, at least 84%, at least 85%, at least 86%, at least 87%, at least 88%, at least 89%, at least 90%, at least 91%, at least 92%, at least 93% at least 94%, at least 95%, at least 96%, at least 97%, at least 98%, at least 99%, at least 99.5% or 100% sequence identity to one of the amino acid sequences specified in SEQ ID NOs 1-27, preferably 1-15, 16-17 or 18-22, more preferably 1-15 or 16-17, in particular 1-15.

In various embodiments, the peptide according to the invention may be used in the detergent or cleaning agent, without being limited thereto, at a concentration of 0.00001 to 5 wt. %.

The detergent or cleaning agent is particularly suitable for use on textiles made of plastic or with a plastic content (blended fabrics). Preferably, the textile comprises or consists of LSEP or an LSEP blended fabric, more preferably polyester, polyethylene (PE), polypropylene (PP), polyurethane (PU), polystyrene (PS), polyvinyl chloride (PVC), polycarbonate (PC), polyamide (PA), polyphenylene ether, polyphenylene sulfide, polyoxymethylene (POM), polymethyl methacrylate (PMA), polyethylene terephthalate (PET), polybutylene terepthalate (PBT), polytetrafluoroethylene (PTFE), polyhydroxyalkanoate (PHA), polyhydroxybutyrate (PHB) polyimide (PI), polylactide (PLA), polyvinylidene fluoride (PVDF), polyether ketone (PEK etc.), and/or copolymers or a blended fabric thereof, even more preferably polyester (PES), polyethylene (PE), polypropylene (PP), polystyrene (PS), copolymers or blended fabrics thereof, preferably a cotton/polyester blend having a polyester content of at least 10%, preferably at least 20%, more preferably at least 30%, even more preferably at least 40%, even more preferably at least 50%, even more preferably at least 60%, for example 65%, or pure polyester or copolymers thereof.

The term “blend” or “blended fabric” refers to textiles having a plastic content, preferably textiles made of at least one natural fiber and at least one plastic fiber (plastic content). In particular, the blend or blended fabric consists of cotton and at least one plastic, in particular polyester. In preferred embodiments, a textile having a plastic content or a plastic blend or blended fabric has a plastic content of at least 10%, more preferably at least 20%, even more preferably at least 30%, even more preferably at least 40%, even more preferably at least 50%, even more preferably at least 60%, for example 65%.

Furthermore, the detergent or cleaning agent according to the invention may contain at least one surfactant.

Anionic, cationic, non-ionic and/or amphoteric surfactants can be used as surfactants in the detergent or cleaning agent according to the invention.

In preferred embodiments, the at least one surfactant is contained in an amount of from 0.5 to 60 wt. %, for example from 0.5 to 50 wt. %, preferably from 0.6 to 40 wt. %, more preferably from 0.7 to 30 wt. %, even more preferably from 0.8 to 20 wt. %, even more preferably from 1 to 15 wt. %, based on the total weight of the detergent or cleaning agent.

In preferred embodiments, the detergent or cleaning agent according to the invention comprises at least one anionic surfactant.

Anionic surfactants are important components in detergent or cleaning agents because they remove a wide range of textile soils and are particularly effective against greasy soils. They are widely available commercially and have good cleaning performance on contaminated surfaces. The surfactants that can be used may be of petrochemical, vegetable or microbiological origin.

Suitable anionic surfactants of the sulfonate type are, for example, C₉-C₁₃ alkyl benzene sulfonates and olefin sulfonates, i.e., mixtures of alkene and hydroxyalkane sulfonates as well as disulfonates, such as those obtained from C₁₂-C₁₈ monoolefins with terminal or internal double bonds by sulfonation with gaseous sulfur trioxide and subsequent alkaline or acid hydrolysis of the sulfonation products. Alkane sulfonates obtained from C₁₂-C₁₈ alkanes, for example by sulfochlorination or sulfoxidation with subsequent hydrolysis or neutralization, are also suitable. The esters of α-sulfofatty acids (ester sulfonates), for example the α-sulfonated methyl esters of hydrogenated coconut, palm kernel or tallow fatty acids, are also suitable.

Other suitable anionic surfactants are sulfated fatty acid glycerol esters. Fatty acid glycerol esters shall be understood to mean the monoesters, diesters and triesters and the mixtures thereof, as they are obtained during production by way of the esterification of a monoglycerol with 1 to 3 moles fatty acid or during the transesterification of triglycerides with 0.3 to 2 moles glycerol. Preferred sulfated fatty acid glycerol esters are the sulfation products of saturated fatty acids having 6 to 22 carbon atoms, for example caproic acid, caprylic acid, capric acid, myristic acid, lauric acid, palmitic acid, stearic acid or behenic acid.

The alkali salts and in particular the sodium salts of the sulfuric acid half-esters of C12-C18 fatty alcohols, for example from coconut fatty alcohol, tallow fatty alcohol, lauryl alcohol, myristyl alcohol, cetyl alcohol or stearyl alcohol, or of C10-C20 oxo alcohols and the half-esters of secondary alcohols having this chain length are preferred as alk(en)yl sulfates. Further preferred are alk(en)yl sulfates of the chain length mentioned, which contain a synthetic straight-chain alkyl residue produced on a petrochemical basis and which have a degradation behavior analogous to that of the adequate compounds based on fat chemical raw materials. For washing reasons, the C12-C16 alkyl sulfates and C12-C15 alkyl sulfates as well as C14-C15 alkyl sulfates are preferred. 2,3-alkyl sulfates are also suitable anion surfactants.

The sulfuric acid monoesters of straight-chain or branched C7-C21 alcohols ethoxylated with 1 to 6 moles of ethylene oxide, such as 2-methyl-branched C9-C11 alcohols having an average of 3.5 moles of ethylene oxide (EO) or C12-C18 fatty alcohols having 1 to 4 EO, are also suitable. It is even preferred that the detergent or cleaning agent according to the invention contains 0.01 to 5 wt. % of an ethoxylated fatty alcohol sulfate.

Further suitable anionic surfactants are also the salts of alkyl sulfosuccinic acid, which are also referred to as sulfosuccinates or as sulfosuccinic acid esters and represent monoesters and/or diesters of sulfosuccinic acid with alcohols, preferably fatty alcohols, and in particular ethoxylated fatty alcohols. Preferred sulfosuccinates contain C8-C18 fatty alcohol residues or mixtures thereof. Particularly preferred sulfosuccinates contain a fatty alcohol residue which is derived from ethoxylated fatty alcohols. In turn, sulfosuccinates whose fatty alcohol residues are derived from ethoxylated fatty alcohols with a narrow homolog distribution are particularly preferred. Similarly, it is also possible to utilize alk(en)yl succinic acid with preferably 8 to 18 carbon atoms in the alk(en)yl chain or salts thereof.

If a fatty acid soap is contained in the detergent or cleaning agent, saturated and unsaturated fatty acid soaps, such as the salts of lauric acid, myristic acid, palmitic acid, stearic acid, (hydrogenated) erucic acid and behenic acid, as well as soap mixtures derived in particular from natural fatty acids, for example coconut, palm kernel, olive oil or tallow fatty acids, are suitable.

The anionic surfactants including soaps, if present, may preferably be in the form of their sodium, potassium or magnesium salts. Preferably, the anionic surfactants are present in the form of their sodium salts.

In various embodiments, the at least one surfactant may comprise an anionic surfactant, for example, but not limited to, a linear or branched alkyl benzene sulfonate and/or a sodium lauryl ether sulfate and/or alpha-olefin sulfonate.

In various embodiments, the at least one surfactant comprises a non-ionic surfactant, for example, without being limited thereto, a non-ionic surfactant selected from the group consisting of alkoxylated fatty alcohols, alkoxylated fatty acid alkyl esters, fatty acid amides, alkoxylated fatty acid amides, polyhydroxy fatty acid amides, alkylphenol polyglycol ethers, amine oxides, alkylpolyglucosides and mixtures thereof, more preferably from the group of alcohol ethoxylates, even more preferably a C12-18 alcohol ethoxylate having 7 EO units. The non-ionic surfactant is preferably contained in an amount of from 0.5 to 50 wt. %, more preferably from 0.6 to 40 wt. %, even more preferably from 0.7 to 30 wt. %, even more preferably from 0.8 to 20 wt. %, even more preferably from 1 to 15 wt. %, based on the total weight of the detergent or cleaning agent.

The non-ionic surfactants utilized are preferably alkoxylated, advantageously ethoxylated, in particular primary alcohols having preferably 8 to 18 C atoms and on average 1 to 12 moles of ethylene oxide (EO) per mole of alcohol, in which the alcohol residue can be linear or preferably methyl-branched in the 2-position or can contain linear and methyl-branched residues in the mixtures, as they are usually present in oxo alcohol residues. However, alcohol ethoxylates having linear residues of alcohols of native origin having 12 to 18 C atoms, for example of coconut, palm, tallow fatty or oleyl alcohol and, on average, 2 to 8 EO per mol of alcohol are particularly preferred. Preferred ethoxylated alcohols include, for example, C12-C14 alcohols having 3 EO, 4 EO or 7 EO, C9-C11 alcohols having 7 EO, C13-C15 alcohols having 3 EO, 5 EO, 7 EO or 8 EO, C12-C18 alcohols having 3 EO, 5 EO or 7 EO, and mixtures thereof, such as mixtures of C12-C14 alcohol having 3 EO and C12-C18 alcohol having 7 EO. The degrees of ethoxylation specified represent statistical averages that can correspond to an integer or a fractional number for a specific product. Preferred alcohol ethoxylates have a narrow homolog distribution (narrow range ethoxylates, NRE). In addition to these non-ionic surfactants, fatty alcohols having more than 12 EO can also be used. Examples of these are tallow fatty alcohols having 14 EO, 25 EO, 30 EO, or 40 EO. Non-ionic surfactants that contain EO and PO groups together in the molecule can also be utilized according to the invention. Block copolymers having EO-PO block units or PO-EO block units can be utilized here, but also EO-PO-EO copolymers or PO-EO-PO copolymers. It is of course also possible to utilize mixed alkoxylated non-ionic surfactants in which EO and PO units are not distributed in blocks, but rather statistically. Products of this kind can be obtained by the simultaneous action of ethylene oxide and propylene oxide on fatty alcohols.

In addition, alkyl glucosides of the general formula RO(G)ₓ can also be utilized as further non-ionic surfactants, in which R means a primary straight-chain or methyl-branched, in particular in the 2-position methyl-branched, aliphatic residue having 8 to 22, preferably 12 to 18, carbon atoms, and G is the symbol representing a glycoside unit having 5 or 6 carbon atoms, preferably for glucose. The degree of oligomerization x, which indicates the distribution of monoglycosides and oligoglycosides, is any number between 1 and 10; preferably x is from 1.2 to 1.4.

Another class of preferred non-ionic surfactants, utilized either as sole nonionic surfactant or in combination with other nonionic surfactants, are alkoxylated, preferably ethoxylated or ethoxylated and propoxylated fatty acid alkyl esters, preferably having 1 to 4 carbon atoms in the alkyl chain, in particular fatty acid methyl esters.

Non-ionic surfactants of the amine oxide type, for example N-cocosalkyl-N,N-dimethylamine oxide and N-tallow alkyl-N,N-dihydroxyethylamine oxide, and of fatty acid alkanolamides may also be suitable. The amount of these non-ionic surfactants is preferably no more than that of the ethoxylated fatty alcohols, in particular no more than half thereof.

Other suitable surfactants are polyhydroxy fatty acid amides of formula (I),

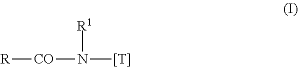

in which RCO represents an aliphatic acyl residue containing 6 to 22 carbon atoms, R¹ represents hydrogen, an alkyl or hydroxyalkyl residue containing 1 to 4 carbon atoms and [T] represents a linear or branched polyhydroxyalkyl residue containing 3 to 10 carbon atoms and 3 to 10 hydroxyl groups. Polyhydroxy fatty acid amides are known substances that can usually be obtained by reductive amination of a reducing sugar with ammonia, an alkylamine or an alkanolamine, and subsequent acylation with a fatty acid, a fatty acid alkyl ester or a fatty acid chloride.

The group of polyhydroxy fatty acid amides also includes compounds of formula (II),

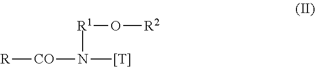

in which R represents a linear or branched alkyl or alkenyl residue containing 7 to 12 carbon atoms, R¹ represents a linear, branched or cyclic alkyl residue or an aryl residue containing 2 to 8 carbon atoms, and R² represents a linear, branched or cyclic alkyl residue or an aryl residue or an oxyalkyl residue containing 1 to 8 carbon atoms, wherein C1-C4 alkyl or phenyl residues are preferred and [T] represents a linear polyhydroxyalkyl residue, the alkyl chain of which is substituted with at least two hydroxyl groups, or alkoxylated, preferably ethoxylated or propoxylated derivatives of this residue. [T] is preferably obtained by reductive amination of a sugar, for example glucose, fructose, maltose, lactose, galactose, mannose or xylose. The N-alkoxy- or N-aryloxy-substituted compounds can then be converted into the desired polyhydroxy fatty acid amides by reaction with fatty acid methyl esters in the presence of an alkoxide as catalyst.

However, it may be preferred that the detergent or cleaning agent has a detergent or cleaning agent matrix based purely on non-ionic surfactants. In particular, with regard to the environmental friendliness of the detergent or cleaning means, it may be preferred that the detergent or cleaning means predominantly contains surfactants from renewable raw materials and that the proportion of synthetic surfactants is as low as possible or is zero.

Suitable cationic surfactants are, inter alia, the quaternary ammonium compounds of the formula (Rⁱ)(Rⁱⁱ)(Rⁱⁱⁱ)(R^{iv})N⁺X⁻, in which Rⁱ to R^{iv} represents four identical or different, in particular two long-chain and two short-chain, alkyl residues and X⁻ for an anion, in particular a halide ion, for example didecyldimethylammonium chloride, alkylbenzyldidecylammonium chloride, alkyl dimethyl hydroxyethylammonium chloride, bromide or methyl sulfate, in particular with C₁₂ alkyl, and mixtures thereof. The detergents or cleaning agents may contain cationic surfactants in amounts, based on the total weight of the detergent or cleaning agent, of 0.001 to 10 wt. %, preferably 0.01 to 5 wt. %, in particular 0.1 to 3 wt. %.

Suitable amphosurfactants (zwitterionic surfactants) are, for example, betaines, alkylamidoalkylamines, alkyl-substituted amino acids, acylated amino acids or biosurfactants, of which betaines are preferred in the teaching according to the invention.

In a preferred embodiment, the detergent or cleaning agent according to the invention contains at least one enzyme.

The detergent or cleaning agent of the present invention may comprise at least one enzyme, or a combination of different enzymes, to provide sufficient cleaning efficacy. Enzymes are preferably used in liquid detergents or cleaning agents, and more preferably in liquid aqueous detergents or cleaning agents.

Enzymes preferably used according to the invention are amylases, proteases, (hemi)cellulases, peroxidases and/or lipases.

Amylases can be added, for example, to remove starch and glycogen. According to the invention, alpha-, beta- and gamma-amylases (α-, β-, γ-amylases), as well as glucoamylases and maltogenic amylases can be used. The amylases can originate from any source, such as bacteria, fungi, pancreatic glands of animal origin, germinated grains, yeasts, etc. Genetically modified amylases can also be used, if necessary even preferably, in the detergents or cleaning agents according to the invention.

The amylase enzymes may be present in the detergents or cleaning agents of the invention in an amount of from 0.00001 wt. % to 5 wt. %, preferably from 0.0001 wt. % to approximately 1 wt. %, more preferably from 0.0005 to approximately 0.5 wt. %, and in particular from 0.01 to approximately 0.4 wt. %, based on the total weight of the detergent or cleaning agent.

In addition to amylases, proteases can also be added to the detergents or cleaning agents according to the invention for cleavage of proteins and peptide residues. Proteases are particularly suitable for hydrolytic cleavage and removal of protein residues, in particular dried protein residues.

Proteases suitable according to the invention are proteinases (endopeptidases) and peptidases (exopeptidases). Usable proteases can be of plant, animal, bacterial and/or fungal origin. Suitable proteases are in particular serine, cysteine, aspartate and metalloproteases. Genetically modified proteases can also be used, if necessary even preferably, in the detergents or cleaning agents according to the invention.

Typically, proteases are utilized in the range of 0.00001 to 1.5 wt. %, preferably in the range of 0.0001 to 0.75 wt. %, based on the total weight of the detergents or cleaning agents.

Furthermore, lipases can be added to detergents or cleaning agents according to the invention to remove firmly adhering fatty soil. Lipases are thus a bio-alternative to surfactants and can support the cleaning effect of surfactants in the range of 0.0001 to 1 wt. %. Suitable lipases can be obtained from plants (for example, rhizine species), microorganisms, and animal sources, such as pancreatic lipases.

The aforementioned enzymes can be added individually or in any combination of mixtures with one another to the detergents and cleaning agents according to the invention. Amylases, in particular alpha-amylases and proteases, are particularly preferred for use in detergents and cleaning agents according to the invention.

The addable enzymes can be combined with any other enzymes, if desired, to further improve the cleaning performance of the detergents or cleaning agents. Other enzymes suitable according to the invention are reductases, oxidases, ligninases, cutinases, pectinases, xylanases, phenoloxidases, lipoxygenases, tannases, pentosanases, malanases, glucanases, arabinosidases and any mixtures of these enzymes.

In a preferred embodiment, the at least one enzyme is used in the detergent or cleaning agent according to the invention in an amount of from 0.00001 to 5 wt. %, preferably from 0.0001 to 2 wt. %, more preferably from 0.01 to 1.5 wt. %, based on the total weight of the detergent or cleaning agent.

Amylases are known to be stabilized by the addition of calcium chloride ions. Boric acid/borates/perborates, in combination with glycerol and/or PEG, as well as niosurfactants with available hydroxyl groups, are other suitable stabilizing agents.

Other suitable additives for the detergent or cleaning agent according to the invention can be selected, for example, from builders, bleaching agents, bleach catalysts, bleach activators, electrolytes, pH adjusting agents, perfumes, perfume carriers, fluorescent agents, dyes, hydrotropes, foam inhibitors, silicone oils, soil release polymers, graying inhibitors, shrinkage inhibitors, wrinkle inhibitors, antimicrobial active ingredients, solvents, germicides, fungicides, antioxidants, preservatives, corrosion inhibitors, antistatic agents, bittering agents, ironing aids, phobizing and impregnating agents, skin-care active ingredients, swelling and anti-slip agents, complexing agents, softening components and UV absorbers and mixtures thereof, in particular solvents such as water and/or organic solvents, surfactants, thickeners, stabilizers, enzymes, fragrances or perfumes, complexing agents, pH adjusting agents, dye transfer inhibitors, antifoaming agents and/or mixtures thereof.

In a preferred embodiment, the detergent or cleaning agent according to the invention comprises the at least one additive in an amount of from 0.0001 to 30 wt. %, preferably from 0.1 to 20 wt. %, more preferably from 1 to 10 wt. %, based on the total weight of the detergent or cleaning agent.

Preferably, the detergent or cleaning agent contains at least one perfume or fragrance, or optionally a mixture of different perfumes or fragrances as the at least one additive.

In the context of the present invention, individual olfactory compounds, e.g., synthetic products of the ester, ether, aldehyde, ketone, alcohol and hydrocarbon type, can be used as perfume oils or fragrances. Preferably, however, mixtures of different fragrances are used, which together create an appealing scent. Such perfume oils may also contain natural mixtures of olfactory substances, such as those available from plant sources, e.g., pine, citrus, jasmine, patchouli, rose or ylang-ylang oils. Suitable fragrances and perfumes or mixtures thereof are known to the person skilled in art in the field of detergent or cleaning agent production.

In a preferred embodiment, the detergent or cleaning agent contains one or more fragrances in an amount of usually up to 15 wt. %, preferably 0.01 to 5 wt. %, in particular 0.3 to 3 wt. %, based on the total weight of the detergent or cleaning agent.

The detergent or cleaning agent according to the invention may further contain one or more builders.

In a further preferred embodiment, the detergent or cleaning agent contains water-soluble and/or water-insoluble builder, in particular selected from alkali aluminosilicate, crystalline alkali silicate with module above 1, monomeric polycarboxylate, polymeric polycarboxylate and mixtures thereof, in particular in amounts in the range of 2.5 wt. % to 30 wt. %, based on the total weight of the detergent or cleaning agent.

The water-soluble organic builder substances include in particular those from the class of polycarboxylic acids, in particular citric acid and sugar acids, and polymeric (poly)carboxylic acids, in particular the polycarboxylates accessible by oxidation of polysaccharides, polymeric acrylic acids, methacrylic acids, maleic acids and copolymers thereof, which may also contain small proportions of polymerizable substances without carboxylic acid functionality in polymerized form. The relative molecular mass of homopolymers of unsaturated carboxylic acids is generally between 5000 g/mol and 200000 g/mol, that of copolymers between 2000 g/mol and 200000 g/mol, preferably 50000 g/mol to 120000 g/mol, based on free acid. A particularly preferred acrylic acid-maleic acid copolymer has a relative molecular mass of from 50,000 g/mol to 100,000 g/mol. Suitable, albeit less preferred compounds of this class are copolymers of acrylic acid or methacrylic acid with vinyl ethers, such as vinyl methyl ethers, vinyl esters, ethylene, propylene, and styrene, in which the proportion of the acid is at least 50 wt. %.

It is also possible to utilize, as water-soluble organic builders, terpolymers which contain two carboxylic acids and/or the salts thereof as monomers and vinyl alcohol and/or a vinyl alcohol derivative or a carbohydrate as the third monomer. The first acidic monomer or salt thereof is derived from a monoethylenically unsaturated C₃-C₈ carboxylic acid and preferably from a C₃-C₄ monocarboxylic acid, in particular from (meth-)acrylic acid. The second acidic monomer or salt thereof may be a derivative of a C₄-C₈ dicarboxylic acid, with maleic acid being particularly preferred. In this case, the third monomeric unit is formed by vinyl alcohol and/or preferably an esterified vinyl alcohol. In particular, vinyl alcohol derivatives are preferred, which are an ester of short-chain carboxylic acids, for example C₁-C₄ carboxylic acids, with vinyl alcohol. Preferred terpolymers contain 60 wt. % to 95 wt. %, in particular 70 wt. % to 90 wt. %, of (meth)acrylic acid and/or (meth)acrylate, more preferably acrylic acid and/or acrylate, and maleic acid and/or maleate, and 5 wt. % to 40 wt. %, preferably 10 wt. % to 30 wt. %, of vinyl alcohol and/or vinyl acetate. Terpolymers in which the weight ratio of (meth)acrylic acid and/or (meth)acrylate to maleic acid and/or maleate is between 1:1 and 4:1, preferably between 2:1 and 3:1 and in particular 2:1 and 2.5:1, are particularly preferred. Both the amounts and the weight ratios are based on the acids. The second acidic monomer or salt thereof may also be a derivative of an allyl sulfonic acid substituted in the 2-position with an alkyl residue, preferably a C₁-C₄ alkyl residue, or an aromatic residue derived preferably from benzene or benzene derivatives. Preferred terpolymers contain 40 wt. % to 60 wt. %, in particular 45 to 55 wt. % of (meth)acrylic acid and/or (meth)acrylate, more preferably acrylic acid and/or acrylate, 10 wt. % to 30 wt. %, preferably 15 wt. % to 25 wt. % of methallylsulfonic acid and/or methallylsulfonate and, as a third monomer, 15 wt. % to 40 wt. %, preferably 20 wt. % to 40 wt. % of a carbohydrate. This carbohydrate may be, for example, a mono-, di-, oligo- or polysaccharide, wherein mono-, di- or oligosaccharides are preferred, sucrose being particularly preferred. The use of the third monomer presumably incorporates predetermined breaking points into the polymer which are responsible for the good biodegradability of the polymer. These terpolymers generally have a relative molecular mass between 1000 g/mol and 200000 g/mol, preferably between 2000 g/mol and 50000 g/mol, and in particular between 3000 g/mol and 10000 g/mol. They can be used, in particular for the preparation of liquid agents, in the form of aqueous solutions, preferably in the form of 30 to 50 wt % aqueous solutions. All of the polycarboxylic acids mentioned are generally used in the form of their water-soluble salts, in particular their alkali salts.

The water-insoluble, water-dispersible inorganic builder materials utilized are, in particular, crystalline or amorphous alkali aluminosilicates, in amounts of up to 50 wt. %, preferably not above 40 wt. %, and in liquid agents, in particular, from 1 wt. % to 5 wt. %. Among these, the crystalline aluminosilicates of detergent grade, in particular zeolite NaA and optionally NaX, are preferred. Amounts close to the upper limit mentioned are preferably used in solid, particulate agents. Suitable aluminosilicates in particular have no particles with a grain size above 30 μm and preferably consist of at least 80 wt. % of particles with a size below 10 μm. Their calcium binding capacity, which can be determined according to the German specification DE 24 12 837 A1, is in the range of 100 to 200 mg CaO per gram. Suitable substitutes or partial substitutes for the aluminosilicate mentioned are crystalline alkali silicates, which may be present alone or in a mixture with amorphous silicates. The alkali silicates useful as builders in the agents preferably have a molar ratio of alkali oxide to SiO₂ below 0.95, in particular from 1:1.1 to 1:12, and may be amorphous or crystalline. Preferred alkali silicates are the sodium silicates, particularly the amorphous sodium silicates, with a molar ratio Na₂O:SiO₂ of 1:2 to 1:2.8. Those with a molar ratio Na₂O:SiO₂ of 1:1.9 to 1:2.8 are preferably added as a solid during production and not in the form of a solution. Crystalline phyllosilicates of the general formula Na₂SiₓO_{2x+1′}yH₂O, where x, referred to as the module, is a number from 1.9 to 4, y is a number from 0 to 20, and preferred values for x are 2, 3 or 4, are preferably used as crystalline silicates, which may be present alone or in a mixture with amorphous silicates. Preferred crystalline phyllosilicates are those in which x assumes the values 2 or 3 in the mentioned general formula. In particular, both β- and δ-sodium disilicates (Na₂Si₂O₅·yH₂O) are preferred. Practically anhydrous crystalline alkali silicates prepared from amorphous alkali silicates of the above-mentioned general formula, in which x is a number from 1.9 to 2.1, can also be used in the agents described herein. In another preferred embodiment of agents according to the invention, a crystalline sodium phyllosilicate having a module of 2 to 3 is used, such as can be produced from sand and soda ash. In another preferred embodiment, crystalline sodium silicates with a module in the range of 1.9 to 3.5 are utilized in detergents. Their content of alkali silicates is preferably 1 wt. % to 50 wt. %, and in particular 5 wt. % to 35 wt. %, based on anhydrous active substance. If alkali alumosilicate, in particular zeolite, is also present as an additional builder substance, the content of alkali silicate is preferably 1 wt. % to 15 wt. %, and in particular 2 wt. % to 8 wt. %, based on anhydrous active substance. The weight ratio of aluminosilicate to silicate, in each case based on anhydrous active substances, is then preferably 4:1 to 10:1. In agents containing both amorphous and crystalline alkali silicates, the weight ratio of amorphous alkali silicate to crystalline alkali silicate is preferably 1:2 to 2:1 and in particular 1:1 to 2:1.

In addition to the inorganic builder mentioned, further water-soluble or water-insoluble inorganic substances may be contained in the agents used together with it or used in the methods according to the invention. Alkali carbonates, alkali hydrogen carbonates and alkali sulfates as well as mixtures thereof are suitable in this context. Such additional inorganic material may be present in amounts up to 30 wt. %.

In preferred embodiments, the detergent or cleaning agent according to the invention may contain at least one further complexing agent. Complexing agents are also known as chelating agents or sequestering agents. Typically, a complexing agent can bind metal ions to prevent them from reacting with other components of a composition. For example, they can be added to detergent or cleaning compositions to complex Ca and Mg ions to soften the water. Other complexing agents may also preferentially contribute to washing or cleaning performance.

Suitable complexing agents include condensed phosphates, phosphonates, and/or aminocarboxylic acids.

Examples of condensed phosphates include, but are not limited to, sodium and potassium orthophosphate, sodium and potassium pyrophosphate, sodium tripolyphosphate, and sodium hexametaphosphate.

Examples of phosphonic acids, phosphonates or derivatives thereof include, but are not limited to, 2-phosphonobutane-1,2,4-tricarboxylic acid (PBTC), 1-hydroxyethane-(1,1-diphosphonic acid) (HEDP), aminotrimethylene-phosphonic acid (ATMP), 2-hydroxyethyliminobis(methylenephosphonic acid), diethylenetriaminepenta(methylenephosphonic acid) (DTPMP), ethylenediaminetetra(methylenephosphonic acid) (EDTMP) hexamethylenediamine(tetramethylenephosphonic acid), bis(hexamethylene)triamine(pentamethylenephosphonic acid), phosphoric acid, or suitable salts thereof.

In a preferred embodiment, the detergent or cleaning agent according to the invention contains 1-hydroxyethane-(1,1-diphosphonic acid) (HEDP) or a suitable salt thereof.

Suitable aminocarboxylic acid materials containing little or no NTA include, but are not limited to, N-hydroxyethylaminodiacetic acid, ethylenediaminetetraacetic acid (EDTA), hydroxyethylenediaminetetraacetic acid, diethylenetriaminepentaacetic acid (DTPA), N-hydroxyethyl-ethylenediaminetriacetic acid (HEDTA), methylglycinediacetic acid (MGDA), glutamic acid-N,N-diacetic acid (GLDA), ethylenediaminesuccinyl acid (EDDS), 2-hydroxyethyliminodiacetic acid (HEIDA), iminodisuccinyl acid (IDS), 3-hydroxy-2-2′-iminodisuccinyl acid (HIDS), and other similar acids or salts thereof having an amino group with a carboxylic acid substituent.

However, in a preferred embodiment, the detergent or cleaning agent is substantially free of aminocarboxylic acids.

If complexing agents should be used in the detergent or cleaning agent according to the invention, they are preferably utilized in an amount of from 0.01 to 30 wt. %, preferably from 0.1 to 20 wt. %, more preferably from 0.2 to 15 wt. %, based on the total weight of the detergent or cleaning agent.

The preferred solvent in the detergent or cleaning agents according to the invention is water, but organic solvents may also be present in the detergent or cleaning agents and partially replace the water.

Suitable organic solvents are, for example, saturated or unsaturated, preferably saturated, branched or unbranched C₁₋₂₀ hydrocarbons, preferably C₂₋₁₅ hydrocarbons, having one or more hydroxy groups, preferably one hydroxy group, and optionally one or more ether functions C—O—C, i.e., oxygen atoms interrupting the carbon atom chain.

Preferred solvents are C₁₋₆ alcohols, in particular ethanol, n-propanol or iso-propanol, as well as C₂₋₆ alkylene glycols and poly-C₂₋₃ alkylene glycol ethers—optionally etherified on one side with a C₁₋₆ alkanol—having on average 1 to 9 identical or different, preferably identical, alkylene glycol groups per molecule, in particular the poly-C₂₋₃ alkylene glycol ethers etherified on one side with a C₁₋₆ alkanol having on average 1 to 9, preferably 2 to 3, ethylene or propylene glycol groups, for example PPG-2 methyl ether (dipropylene glycol monomethyl ether).

Exemplary solvents are the following connections named according to INCI: alcohol (ethanol), buteth-3, butoxydiglycol, butoxyethanol, butoxyisopropanol, butoxypropanol, n-butyl alcohol, t-butyl alcohol, butylene glycol, butyloctanol, diethylene glycol, dimethoxydiglycol, dimethyl ether, dipropylene glycol, ethoxydiglycol, ethoxyethanol, ethyl hexanediol, glycol, hexanediol, 1,2,6-hexanetriol, hexyl alcohol, hexylene glycol, isobutoxypropanol, isopentyldiol, isopropyl alcohol (iso-propanol), 3-methoxybutanol, methoxydiglycol, methoxyethanol, methoxyisopropanol, methoxymethylbutanol, methoxy PEG-10, methylal, methyl alcohol, methyl hexyl ether, methylpropanediol, neopentyl glycol, PEG-4, PEG-6, PEG-7, PEG-8, PEG-9, PEG-6 methyl ether, pentylene glycol, PPG-7, PPG-2-buteth-3, PPG-2 butyl ether, PPG-3 butyl ether, PPG-2 methyl ether, PPG-3 methyl ether, PPG-2 propyl ether, propanediol, propyl alcohol (n-propanol), propylene glycol, propylene glycol butyl ether, propylene glycol propyl ether, tetrahydrofurfuryl alcohol, trimethylhexanol, together with aliphatic or aromatic alcohols, e.g., methanol, ethanol, n-propanol, n-butanol, tert-butanol or phenol, or carboxylic acids, e.g., acetic or carbonic acid, etherified or esterified monomers or homo- or heteropolymers, in particular monomers and homodi- and trimers, C₂-C₄-alkylene glycols.

In a preferred embodiment of the invention, the detergent or cleaning agents contain one or more hydrophobic components. The hydrophobic components not only improve the cleaning effect with respect to hydrophobic contaminants such as grease dirt, but also have a positive effect on phase separation and its reversibility in multiphase detergents or cleaning agents. Suitable hydrophobic components are, for example, dialkyl ethers with identical or different C₄ to C₁₄ alkyl residues, in particular linear dioctyl ether; hydrocarbons having a boiling range of 100 to 300° C., in particular 140 to 280° C., e.g., aliphatic hydrocarbons having a boiling range of 145 to 200° C., isoparaffins having a boiling range of 200 to 260° C.; essential oils, in particular limonene and the pine oil extracted from pine roots and stumps; and also mixtures of these hydrophobic components, in particular mixtures of two or three of the hydrophobic components mentioned. Preferred mixtures of hydrophobic components are mixtures of different dialkyl ethers, of dialkyl ethers and hydrocarbons, of dialkyl ethers and essential oils, of carbohydrates and essential oils, of dialkyl ethers and hydrocarbons and essential oils and of these mixtures. The detergent or cleaning agents may contain hydrophobic components in amounts, based on the total weight of the detergent or cleaning agent, of 0.01 to 20 wt. %, preferably 0.1 to 14 wt. %, more preferably 0.5 to 10 wt. %, even more preferably 0.8 to 7 wt. %.

Where the detergents or cleaning agents according to the invention are formulated in a multiphase formulation, they may contain one or more phase separation aids. In addition to citric acid or citrates, suitable phase separation aids include, for example, alkali metal and alkaline earth metal halides, in particular chlorides and sulfates as well as nitrates, in particular sodium and potassium chloride and sulfate, as well as ammonium chloride and sulfate or mixtures thereof.

As strong electrolytes that increase the ionic strength, such salts support phase separation through the salt effect. Sodium chloride has proven to be particularly effective in this respect, while sodium sulfate and, in particular, magnesium sulfate have a less phase-separating effect. The detergent or cleaning agents may contain phase separation aids in amounts, based on the total weight of the detergent or cleaning agent, of 0.01 to 30 wt. %, preferably 0.1 to 20 wt. %, for example 3 to 15 wt. % or 5 to 12 wt. %.

To adjust viscosity, the detergent or cleaning agent according to the invention may contain one or more thickening agents, preferably in an amount of from 0.01 to 5 wt %, more preferably from 0.05 to 2.5 wt %, even more preferably from 0.1 to 1 wt %.

Suitable thickeners include organic natural thickeners (agar-agar, carrageenan, tragacanth, gum arabic, alginates, pectins, polyoses, guar flour, carob seed flour, starch, dextrins, gelatin, casein), organic modified natural substances (carboxymethylcellulose and other cellulose ethers, hydroxyethyl and -propyl cellulose and the like, seed flour ethers), organic fully synthetic thickeners (polyacrylic and polymethacrylic compounds, vinyl polymers, polycarboxylic acids, polyethers, polyimines, polyamides), and inorganic thickeners (polysilicas, clay minerals such as montmorillonites, zeolites, silicas).

Polyacrylic and polymethacrylic compounds include, for example, the high-molecular homopolymers of acrylic acid crosslinked with a polyalkenyl polyether, in particular an allyl ether of pentaerythritol or propylene (INCI designation according to the International Dictionary of Cosmetic Ingredients of The Cosmetic, Toiletry, and Fragrance Association (CTFA): Carbomer), which are also referred to as carboxyvinyl polymers. Furthermore, the following acrylic acid copolymers are included: (i) copolymers of two or more monomers from the group of acrylic acid, methacrylic acid and their simple esters, preferably formed with C₁₋₄ alkanols (INCI Acrylates Copolymer), which include for example the copolymers of methacrylic acid, butyl acrylate and methyl methacrylate (CAS designation according to the Chemical Abstracts Service: 25035-69-2) or of butyl acrylate and methyl methacrylate (CAS 25852-37-3); (ii) crosslinked high-molecular acrylic acid copolymers, which include for example the copolymers of C10-30 alkyl acrylates crosslinked with an allyl ether of pentaerythritol having one or more monomers selected from the group consisting of acrylic acid, methacrylic acid and their simple esters, preferably formed with C₁₋₄ alkanols (INCI Acrylates/C10-30 Alkyl Acrylate Crosspolymer). In addition to the thickening effect, these compounds in detergents can have other effects, such as protection against graying.

In a preferred embodiment, the polyacrylic and polymethacrylic compounds suitable as thickeners have a weight average molecular weight of >100,000 g/mol, preferably <500,000 g/mol.

Preferred thickening agents are the polysaccharides and heteropolysaccharides, in particular the polysaccharide gums, for example gum arabic, agar, alginates, carrageenans and their salts, guar, guaran, tragacanth, gellan, ramsan, dextran or xanthan, and their derivatives, e.g., propoxylated guar, as well as their mixtures. Other polysaccharide thickeners, such as starches or cellulose derivatives, can be utilized alternatively, but preferably in addition to a polysaccharide gum, for example starches of various origins and starch derivatives, e.g., hydroxyethyl starch, starch phosphate esters or starch acetates, or carboxymethyl cellulose or its sodium salt, methyl, ethyl, hydroxyethyl, hydroxypropyl, hydroxypropyl methyl or hydroxyethyl methyl cellulose or cellulose acetate.

Polysaccharides and heteropolysaccharides suitable as thickeners preferably have a weight average molecular weight of >1,500 g/mol, more preferably of >5,000 g/mol, even more preferably of >50,000 g/mol. In general, their weight average molecular weight is <250,000 g/mol.

A particularly preferred polymer is the microbial anionic heteropolysaccharide xanthan gum produced by Xanthomonas campestris and some other species under aerobic conditions with a molecular weight of 2 to 15×10⁶ g/mol.

In a preferred embodiment, the detergent or cleaning agent according to the invention is present in liquid, gel or powder form, preferably in liquid, aqueous form.

Preferably, the detergent or cleaning agent according to the invention is a detergent, a laundry after-treatment agent or a laundry care agent, more preferably a detergent.

In a preferred embodiment, the detergents or cleaning agents according to the invention are liquid detergents or cleaning agents, more preferably liquid, aqueous detergents or cleaning agents, even more preferably single-phase, liquid, aqueous detergents or cleaning agents.

Further, an insoluble solid component may also be present as a separate solid phase in single-phase detergent or cleaning agents. When such detergents or cleaning agents according to the invention are shaken, an emulsion of the liquid phase is temporarily formed, which disperses the solid phase within itself.

Multiphase formulations are not preferred in the context of the present invention, but are not excluded.

“Aqueous” in the context of the present invention means that water is the main solvent in the detergent or cleaning agent according to the invention.

However, the detergent or cleaning agent may contain water-soluble organic solvents, such as alcohols, in addition to water.

The term “liquid” preferably refers to a composition that is flowable at room temperature (approx. 20° C.) and ambient pressure (approx. 1013 mbar at sea level). The term may also include gel and paste compositions.

The viscosity of the liquid detergent or cleaning agent at 20° C. is preferably 5 to 100,000 mPa·s, more preferably 10 to 5000 mPa·s, even more preferably 10 to 200 mPa·s, measured with a Brookfield type LVT or LVDV-II+ rotational viscometer with a small sample adapter at a speed of 30 min⁻¹, wherein the Brookfield spindle used as measuring body is to be selected such that the torque is in a favorable range and the measuring range is not exceeded. In this context, spindle 31 is preferred and—if required for viscosities above approximately 240 mPa·s—spindle 25 is preferred.

The pH of the detergent or cleaning agent according to the invention is preferably 2 to 12, preferably 5 to 9, more preferably 7 to 9, both in concentrated form and in diluted application solution.

To adjust such a pH, acids can be added to the detergents or cleaning agents according to the invention. Suitable are inorganic acids, for example the mineral acids, e.g., hydrochloric acid, and organic acids, for example saturated or unsaturated C₁-C₆ mono-, di- and tri-carboxylic acids and hydroxycarboxylic acids with one or more hydroxyl groups, such as citric acid, maleic acid, formic acid and acetic acid, aminosulfuric acid, C₆-C₂₂ fatty acids and anion-active sulfonic acids, and mixtures thereof. Particularly preferred acids are citric acid, more preferably utilized in the form of its monohydrate citric acid×1H₂O, and the anion-active sulfonic acids, as well as combinations of citric acid with one or more anion-active sulfonic acids, in particular with alkylarinsulfonic acids. The citric acid advantageously combines acid phase separation aid and builder properties, while the anion-active sulfonic acids simultaneously act as an acid and anionic surfactant.

In addition, one or more alkalis can be used, for example alkali metal, alkaline earth metal and ammonium hydroxides as well as carbonates and ammonia or amines, preferably sodium and potassium hydroxide and alkanolamines, with monoethanolamine being particularly preferred.

Since, for example, pH-value-changing substances are often introduced into the washing or cleaning liquor in large quantities during the washing or cleaning process, it is preferable to add appropriate buffer substances to the detergent or cleaning agent according to the invention in the application dilution, for example acetates, hydrogen phosphates, hydrogen sulfates, soda or alkali metal bicarbonates, in order to stabilize or buffer the pH value. Particularly suitable buffer systems are potassium hydrogen phthalate/sodium hydroxide, potassium dihydrogen phosphate/sodium hydroxide and the like.

However, within the scope of the present invention, solid detergent or cleaning agents are also included. A substance is described as “solid” if it is in the solid state at room temperature (approx. 20° C.) and ambient pressure (approx. 1013 mbar at sea level).

The detergents or cleaning agents according to the invention, especially if they are liquid or pasty, can be produced by simply mixing the ingredients in an automatic mixer.

In a preferred embodiment, the agents are present, preferably in liquid, gel or paste form, preferably as a portion in a fully or partially water-soluble wrapping, more preferably in single-use portions. The portioning makes it easier for the consumer to dose.

The agents can be packed in film bags, for example. Pouch packaging made of water-soluble film makes it unnecessary for the consumer to tear open the packaging. In this manner, convenient dosing of a single portion measured for one wash cycle is possible by placing the bag directly into the washing machine or by dropping the bag into a specified amount of water, for example, into a bucket, bowl or hand wash basin. The film bag surrounding the washing portion dissolves without leaving any residue when a certain temperature is reached.

In the prior art, there are numerous methods for the production of water-soluble detergent portions, which are in principle also useful in the context of the present invention.

However, the detergent or cleaning agent according to the invention can also be provided unportioned in liquid, gel, paste or solid form in storage bottles or packets.

The detergents or cleaning agents according to the invention can be used for washing and/or cleaning and/or treating textiles made of plastic or having a plastic content. Preferably, the invention relates to detergents for cleaning or treating textiles made of plastic or having a plastic content.

In preferred embodiments, the detergents or cleaning agents according to the invention may comprise further additives.

The general production and composition of detergents and cleaning agents is basically known to the person skilled in the art in this field and can be carried out by any suitable method.

Exemplary detergent and cleaning compositions and suitable ingredients are described, for example, in WO 01/44433 A1 or WO 2016/091650 A1.

An exemplary composition of a detergent according to the invention, in particular a liquid detergent, comprises the following components in addition to the peptides according to the invention:
water; and
surfactants, in particular anionic and/or non-ionic surfactants; and/or
thickeners; and/or
stabilizers; and/or
organic solvents; and/or
enzymes; and/or
fragrances; and/or
complexing agents, for example phosphonates, in particular HEDP; and/or
pH adjusting agents, in particular citric acid, NaOH and/or monoethanolamine (MEA); and/or
dye transfer inhibitors, in particular DTI; and/or
soil release polymers; and/or
antifoaming agent.

Particularly preferably, a detergent or cleaning agent according to the invention comprises, in addition to the peptides according to the invention, the following components:
at least one surfactant, preferably in a concentration of 0.5 to 60 wt. %, in particular 1 to 45 wt. %; and/or
at least one stabilizer, preferably in a concentration of 0.001 to 20 wt. %; and/or
at least one complexing agent, preferably in a concentration of 0.01 to 30 wt. %, in particular 0.1 to 10 wt. %; and/or
at least one organic solvent, preferably in a concentration of 0.1 to 30 wt. %, in particular 0.5 to 25 wt. %; and/or
at least one soil release polymer, preferably in a concentration of 0.01 to 20 wt. %, in particular 0.05 to 10 wt. %; and/or
at least one enzyme, preferably in a concentration of 0.00001 to 5 wt. %; and/or
at least one fragrance or perfume substance and/or at least one fragrance storage substance, preferably in a concentration of 0.0001 to 15 wt. %; and/or
pH adjusting agent, for example MEA, NaOH or citric acid, preferably in a concentration of 0.01 to 25 wt %; and/or
at least one antifoaming agent; and/or
water;
   wherein the concentration information is always based on the total weight of the detergent or cleaning agent.

In a preferred embodiment, the peptide according to the invention in the detergent or cleaning agent may be functionally modified, i.e., conjugated, for example covalently linked, for example with another molecule or chemical group, in particular a (macro)molecule.

In preferred embodiments, the peptide according to the invention can act in the detergent or cleaning agent as an adhesion promoter to coat the textiles with chemical groups or specific molecules. For example, the coating of the textiles can increase the hydrophilicity of the textile and thus cause improved moisture regulation.

Another aspect of the invention relates to a method for cleaning and/or treating textiles that are made of plastic or consist to some extent of plastic, wherein at least one peptide, polypeptide or agent according to the invention is used in at least one method step.

Another aspect of the present invention relates to the use of the at least one peptide or polypeptide (multimer) according to the invention or the agent according to the invention for adhesion to plastic surfaces, in particular LSEP surfaces, as described herein, for example polyester, polyethylene, polypropylene or polystyrene and/or copolymers and/or mixtures thereof. The peptide can comprise or contain an amino acid sequence having at least 80%, preferably at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or 100% sequence identity to one of the amino acid sequences specified in SEQ ID NOs: 1-27, preferably 1-15, 16-17, 18-22 and/or 23-27, more preferably 1-15, 16-17 and/or 18-22, even more preferably 1-15 and/or 16-17, in particular 1-15, for example 2, 5 and 7. In all of these uses, the peptide may be a component of a larger molecule in which it acts as an adhesion promoter.

In a further aspect, the invention relates to the use of the at least one peptide or polypeptide (multimer) according to the invention or the agent according to the invention for cleaning and/or treating textiles that are made of plastic or consist to some extent of plastic, in particular for improved cleaning and/or for improving the wearing comfort of textiles after washing, preferably LSEP plastic textiles, in particular textiles made of a cotton/polyester blend or of polyester or copolymers thereof. The peptide can comprise or contain an amino acid sequence having at least 80%, preferably at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or 100% sequence identity to one of the amino acid sequences specified in SEQ ID NOs: 1-27, preferably 1-15, 16-17, 18-22 and/or 23-27, more preferably 1-15, 16-17 and/or 18-22, even more preferably 1-15 and/or 16-17, in particular 1-15, for example 2, 5 and 7. In these uses too, the peptide can be a component of a larger molecule in which it acts as an adhesion promoter.

The improvement in wearing comfort is preferably reflected in an improved haptic of the textile, an increased soil-repellent effect and/or a changed water absorption or moisture regulation.

In various embodiments, the peptide is coupled to another molecule in the uses according to the invention, as described herein.

In particular, the use of the peptide according to the invention, for example coupled with another molecule or chemical group, for example with a (macro)molecule, allows improved cleaning and/or the textiles are changed, for example, by the adhesion of the peptide of the invention, for example by coating the textile. In this context, the peptide according to the invention serves, for example, as an adhesion promoter in order to apply a coating or certain chemical groups to the surface of the textile, for example. Furthermore, the peptide can serve as an anchor, for example, to bring a particular molecule into spatial proximity to the textile.

The following figures are included in this application:

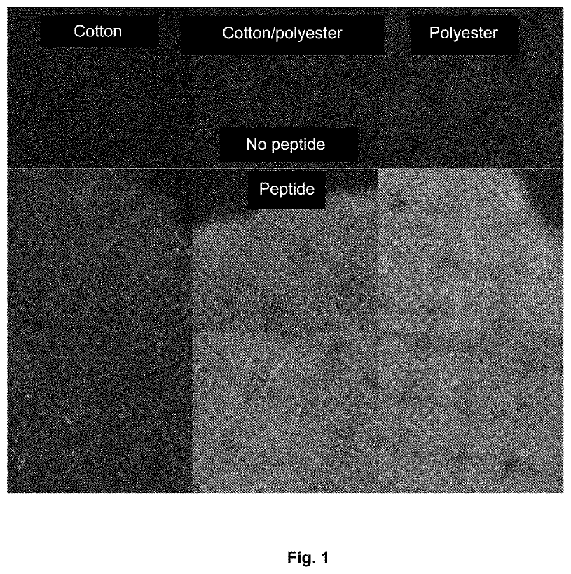

FIG. 1: The fabrics were coated with P8_S8 peptide and stained with Alexa Fluor® 488-streptavidin dye (excitation/emission wavelength maxima of 495 and 519 nm, respectively). Fluorescence images were acquired using an Axio Imager M2 microscope (Zeiss) with objective magnification 10, brightness index 50, exposure time 100 ms, and white balance 5500 K. WFK Testgewebe GmbH (from left to right in the picture): Cotton 10A, 65% Polyester/35% Cotton 20A, Polyester 30A; P8_S8 peptide solution: not applied (blank with all reagents, without peptide) (top row); applied to the fabrics (bottom row).

### EXAMPLES

### Example 1: Peptide Sequences

TABLE 1
Peptide names and associated amino acid sequences.

| No. | Peptide name | Amino acid sequence |
|---|---|---|
| 1 | BA1-A06 | GLHTSATNLYLH (SEQ ID |
| | | NO: 18) |
| 2 | D2 | QHSIRLLTIKKP (SEQ ID |
| | | NO: 19) |
| 3 | D4 | QQSIRIMTIKHP (SEQ ID |
| | | NO: 20) |
| 4 | P3 | WRHPRLRCGNLL (SEQ |
| | | ID NO: 21) |
| 5 | P6 | SRARLFVVTYHK (SEQ ID |
| | | NO: 23) |
| 6 | P7 | HMISTMNAASRR (SEQ ID |
| | | NO: 24) |
| 7 | P8 | RSIVTFSLRQNR (SEQ ID |
| | | NO: 25) |
| 8 | P8_S2 | RSIVTFSLRQNAQLA |
| | | (SEQ ID NO: 16) |
| 9 | P8_S8 | RSIVTFSLRQNSEQA |
| | | (SEQ ID NO: 17) |
| 10 | P13 | RNTIRIRTIKHP (SEQ ID |
| | | NO: 26) |
| 11 | P14 | QKSRNRMTRTHP (SEQ |
| | | ID NO: 22) |
| 12 | P15 | RHSSTLRYRPLP (SEQ ID |
| | | NO: 27) |
| 13 | RAL-M | RALQALRALQALEAL |
| | | (SEQ ID NO: 1) |
| 14 | RAL2E | RALRALRALEALEAL (SEQ |
| | | ID NO: 2) |
| 15 | RAL2Q | RALRALRALQALQAL |
| | | (SEQ ID NO: 3) |
| 16 | RALQE | RALRALRALQALEAL |
| | | (SEQ ID NO: 4) |
| 17 | RALQ2E | RALRALQALEALEAL |
| | | (SEQ ID NO: 5) |
| 18 | RAL2FQ | RALFEALQALFRALEAL |
| | | (SEQ ID NO: 6) |
| 19 | RALEQE | RALRALEALQALEA (SEQ |
| | | ID NO: 7) |
| 20 | RAL2FR | RALFEALFRALEALR (SEQ |
| | | ID NO: 8) |
| 21 | RAL2F | RALFEALFRALEAL (SEQ |
| | | ID NO: 9) |
| 22 | RALF | RALEALFRALEAL (SEQ ID |
| | | NO: 10) |
| 23 | RALF_sym | RALRALFEALEAL (SEQ ID |
| | | NO: 11) |
| 24 | RALF_Q | RALEALFRALQALEAL |
| | | (SEQ ID NO: 12) |
| 25 | RALFQ_W | RALEALWRALQALEAL |
| | | (SEQ ID NO: 13) |
| 26 | RALF_W | RALEALWRALEAL (SEQ |
| | | ID NO: 14) |
| 27 | RALA4Q | RALARALARALAQALA |
| | | (SEQ ID NO: 15) |

The peptides originate in part from E. coli display database screenings (e.g., P3, P6, P15, P8C); other peptides are either further developments thereof or pure designer peptides (for example RAL peptides or variants thereof). The peptide can be present as listed or, for example, can be supplemented at the N- and/or C-terminal by another amino acid such as cysteine. Furthermore, the peptide can, for example, be coupled to another peptide or molecule such as biotin (functionalized peptide). For example, P3 represents the peptide sequence without cysteine, P3C or 1-C represents the peptide sequence with C-terminal cysteine, P3-Bt represents the peptide sequence with C-terminal biotin. Furthermore, the peptide can also occur as a subunit (module) in a polypeptide.

TABLE 2
Charge of the peptide regions.

| No. | Amino acid sequence |
|---|---|
| 8 | RSIVTFSLRQNAQLA (SEQ ID NO: 16) |
| 9 | RSIVTFSLRQNSEQA (SEQ ID NO: 17) |
| 13 | RALQALRALQALEAL (SEQ ID NO: 1) |
| 14 | RALRALRALEALEAL (SEQ ID NO: 2) |
| 15 | RALRALRALQALQAL (SEQ ID NO: 3) |
| 16 | RALRALRALQALEAL (SEQ ID NO: 4) |
| 17 | RALRALQALEALEAL (SEQ ID NO: 5) |
| 19 | RALRALEALQALEAL (SEQ ID NO: 7) |

With reference to Table 2, underlined amino acids denote parts of the peptide (tripeptides) with a positive charge, bold amino acids denote regions of the peptide which are uncharged, and not highlighted amino acids denote parts of the peptide (tripeptides) with a negative charge.

### Example 2: Testing the Adhesion of the Synthesized Peptides with Regard to their Substrate Specificity on Polyester (PES), Blended Fabrics (Polyester/Cotton) and Cotton

### Test Setup:

Direct measurement method with μBCA in 48-well dish with fabric lobules with a diameter of 10 mm:

3 lobules per peptide dilution were placed in the 48-well dish and 0.2 ml of peptide solution (0.02 mg/ml in Aqua dest.) was added to each. It was incubated for 1 h at RT, shaking at 750 rpm. Subsequently, washed 3 times with 0.5 ml Aqua dest., shaking at 750 rpm for 5 min. Then, 0.2 ml of Aqua dest. was added. +0.2 ml BCA reagent was added and incubated for 60 min at 60° C. The absorbance was measured at 562 nm. An adhesion point after infinite adhesion time can be read from the measured adhesion values.

The following peptides were tested on all three surfaces:
P8_S8 with C-terminal cysteine
RAL2FR with C-terminal cysteine
RALP_Q with C-terminal cysteine and

MS-Si (ATIHDAFYSAPE) (SEQ ID NO: 55)—steel-binding peptide as a negative control (Zuo R., Ōrnek, D., Wood, T. K. (2005) Aluminum- and mild steel-binding peptides from phage display, Appl. Microbiol. Biotechnol., 68: 505-509).

The remaining peptides were tested only on polyester fabrics.

AP in % shows how much % of the applied amount (100%) remains on the PIES after washing. The higher the AP value, the higher the binding affinity and thus the adhesion.

TABLE 3
Adhesion test results.

| | | Material | | |
|---|---|---|---|---|
| Peptide | | | Polyester/ | |
| Ident | | Polyester | Cotton | Cotton |
| no. | Name | (PES) | (PES/CO) | (CO) |

LSEP binding peptide

| | | | | |
|---|---|---|---|---|
| 1 | BA1-A06 | 16 | — | — |
| 2 | D2 | 43 | — | — |
| 3 | D4 | 26 | — | — |
| 4 | P3 | 57 | — | — |
| 5-C | P6 | 62 | — | — |
| 6-C | P7 | 38 | — | — |
| 7-C | P8 | 68 | — | — |
| 8-C | P8_S2 | 66 | — | — |
| 9-C | P8_S8 | 62 | 28 | 0 |
| 10 | P13 | 36 | — | — |
| 11 | P14 | 42 | — | — |
| 12 | P15 | 53 | — | — |
| 13-C | RAL-M | 55 | — | — |
| 14-C | RAL2E | 67 | — | — |
| 17-C | RALQ2E | 60 | — | — |
| 18-C | RAL2FQ | 43 | — | — |
| 19-C | RALEQE | 66 | — | — |
| 20-C | RAL2FR | 54 | 3 | 0 |
| 21-C | RAL2F | 33 | — | — |
| 24-C | RALF_Q | 58 | 13 | 0 |
| 25-C | RALFQ_W | 51 | — | — |
| 26-C | RALF_W | 57 | — | — |
| 27-C | RALA4Q | 46 | — | — |

Negative control

| | | | |
|---|---|---|---|
| MS-S1 | 4 | 8 | 1 |

Peptides 5-C, 7-C, 8-C, 9-C, 14-C, 17-C, and 19-C show very good binding affinity to polyester. Mean binding affinities to polyester were shown for peptides 2, 4, 11, 12, 13-C, 18-C, 20-C, and 24-C to 27-C.

### Example 3: Peptide Detection on Textiles by Means of Fluorescence

P8_S8 peptide with C-terminal cysteine is present biotinylated, so that it forms a detectable conjugate after reaction with streptavidin—Alexa Fluor 488.

The fabrics were coated with P8_S8 peptide and stained with Alexa Fluor® 488-streptavidin dye (excitation/emission wavelength maxima of 495 and 519 nm, respectively). Fluorescence images were acquired using an Axio Imager M2 microscope (Zeiss) with objective magnification 10, brightness index 50, exposure time 100 ms, and white balance 5500 K. Between cotton, blended fabric, and polyester, the binding of the adhesive peptide P8CS8 to the polyester surface is the strongest. The negative control confirms that the binding of P8_S8 to polyester is very specific (FIG. 1).

### Example 4: Liquid Detergent Matrix

TABLE 4
Exemplary liquid detergent matrix.

| | Concentration of the | wt. % in the |
|---|---|---|
| Component | starting material | formulation |
| Water demineralized | 100.0 | Fill up |
| | | to 100% |
| Citric acid | 100.0 | 1-5% |
| Stabilizer | 100.0 | 0.3-2% |
| Anti-foaming agents | 100.0 | <1% |
| FAEOS | 70.0 | 3-8% |
| FAEO, Non-ionic surfactant | 100.0 | 2-11% |
| LAS | 96.0 | 3-20% |
| Palm kernel oil fatty acid | 30.0 | 0.3-4% |
| MEA | 100.0 | 4-8% |
| NaOH | 50.0 | 0.5-2% |
| Glycerol | 99.5 | 1-3% |
| Propanediol-1,2 | 100.0 | 8-12% |
| HEDP | 60.0 | 0.5-2% |
| SRP | 30.0 | 0.5-1% |
| optionally enzymes, perfumes, DTI, | t.q. | Traces |
| peptides according to the invention | | |

1. A peptide for adhesion to textiles made of plastic or having a plastic content, comprising or consisting of an amino acid sequence of from 4 to 50 amino acids, preferably from 10 to 24 amino acids, where
(a) the amino acid sequence in N- to C-terminal orientation has the following sequence
(C)ₘX₁X₂X₃(X₄)ₙX₅(C)ₒ,
where
X₁ is a positively charged amino acid, preferably R or K, more preferably R,
X₂ and X₃ are uncharged amino acids, preferably selected from A, L, S, I, M and Q, more preferably A, S, I and L, in particular A and L,
each X₄ is independently of one another any amino acid, preferably with the exception of P, more preferably with the exception of P and G;
X₅ is any positively charged or uncharged amino acid, preferably R or an uncharged amino acid, more preferably Q, A or L, in particular A or L,
m and o are 0 or 1, where m+o=0 or 1; and
n is an integer from 0 to 46, preferably from 6 to 20;
or
(b) the amino acid sequence has at least 80%, preferably at least 85%, more preferably at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or 100% sequence identity to one of the amino acid sequences specified in SEQ ID NOs: 16-17 or 18-22.

2. The peptide according to claim 1, where
(i) the peptide has a total charge of 0 to +4, preferably 0 to +2; and/or
(ii) the N-terminus comprising the first 3-4 amino acids has a net positive charge; and/or
(iii) the C-terminus comprising the last 3-6 amino acids has a net negative or neutral charge and preferably comprises at least one negatively charged amino acid, in particular E; and/or
(iv) the peptide contains no P and preferably also no G and even more preferably also no Y.

3. The peptide according to claim 1, where the sequence X₁X₂X₃ is RAL or RLA, preferably RAL.

4. The peptide according to claim 1, where
(i) (X₄)ₙX₅ comprises at least one sequence X₆X₇X₈, where X₆ is a charged or uncharged amino acid, preferably R, K, E or Q, and X₇ and X₈ independently of one another are uncharged amino acid with the exception of P and G, preferably A, L or M, more preferably A or L, in particular A or L;
(ii) (X₄)ₙ comprises at least one aromatic amino acid, preferably W or F.

5. The peptide according to claim 4, where
(i) (X₄)ₙ comprises at least one sequence X₆X₇X₈, where X₆X₇X₈ is RAL or RLA, preferably RAL, and where said sequence is preferably localized in the N-terminal amino acids of positions 4-7; and/or
(ii) (X₄)ₙX₅ comprises at least one sequence X₆X₇X₈, where X₆X₇X₈ is EAL or ELA, preferably EAL, and where said sequence is preferably not localized in the N-terminal amino acids of positions 1-6; and/or
(iii) (X₄)ₙX₅ comprises at least one sequence X₆X₇X₈, where X₆X₇X₈ is QAL or QLA, preferably QAL.

6. The peptide according to claim 5, where the peptide comprises the sequence X₁X₂X₃ RAL and (X₄)ₙX₅ comprises at least one of QAL and EAL, preferably both.

7. The peptide according to claim 6, where the peptide additionally comprises at least one further sequence RAL.

8. The peptide according to claim 6, where the peptide contains two sequences RAL and at least one sequence each of EAL and QAL.

9. The peptide according to claim 6, where the peptide contains at least one W or F, preferably exactly one W or F.

10. The peptide according to claim 1, where the peptide consists of at least 60%, preferably at least 65%, more preferably at least 70%, in particular at least 75% of amino acids having a high alpha-helix-forming potential, with said amino acids preferably being selected from E, A, L, M, Q, K, R, F, I, H, W and D, more preferably E, A, L, M, Q, K, R, F, I and H; even more preferably E, A, L, M, Q, K, R and F.

11. The peptide according to claim 1, where the peptide has the amino acid sequence according to one of SEQ ID NOs: 1-15 and/or 16-17, as well as variants thereof, which have at least 80%, preferably at least 90%, sequence identity to the specified sequence, with preferably the RAL motif, and preferably also the EAL and/or QAL motif, if present, being invariable.

12. The peptide according to claim 1, where the peptide for adhesion to textiles made of plastic or having a plastic content adheres to textiles made of LSEP plastic or having an LSEP plastic content, in particular to textiles made of polyester or having a polyester content (blended fabric), or copolymers thereof.

13. A peptide or polypeptide (multimer) comprising two or more of the peptides according to claim 1.

14. A detergent or cleaning agent, preferably a detergent, comprising
(a) at least one peptide according to claim 1; or
(b) at least one peptide comprising or consisting of an amino acid sequence having at least 80%, preferably at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or 100% sequence identity to one of the amino acid sequences specified in SEQ ID NOs: 1-27, preferably 1-15, 16-17 or 18-22, more preferably 1-15 or 16-17, in particular 1-15.

15. The detergent or cleaning agent according to claim 14, where the detergent or cleaning agent further comprises at least one, preferably more, of the following components:
at least one surfactant, preferably in a concentration of 0.5 to 60 wt. %, in particular 1 to 45 wt. %; and/or
at least one stabilizer, preferably in a concentration of 0.001 to 20 wt. %; and/or
at least one complexing agent, preferably in a concentration of 0.01 to 30 wt. %, in particular 0.1 to 10 wt. %; and/or
at least one organic solvent, preferably in a concentration of 0.1 to 30 wt. %, in particular 0.5 to 25 wt. %; and/or
at least one soil release polymer, preferably in a concentration of 0.01 to 20 wt. %, in particular 0.05 to 10 wt. %; and/or
at least one enzyme, preferably in a concentration of 0.00001 to 5 wt. %; and/or
at least one fragrance or perfume substance and/or at least one fragrance storage substance, preferably in a concentration of 0.0001 to 15 wt. %; and/or
at least one pH adjusting agent, for example MEA, NaOH or citric acid, preferably in a concentration of 0.01 to 25 wt %; and/or
at least one antifoaming agent; and/or
water;
wherein the concentration information is always based on the total weight of the detergent or cleaning agent.

16. A use of
(a) at least one peptide according to claim 1; or
(b) at least one peptide comprising or consisting of an amino acid sequence having at least 80%, preferably at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or 100% sequence identity to one of the amino acid sequences specified in SEQ ID NOs: 1-27, preferably 1-15, 16-17 or 18-22, more preferably 1-15 or 16-17, in particular 1-15, or of a detergent or cleaning agent comprising at least one peptide thereof;
for cleaning and/or treating textiles that are made of plastic or consist to some extent of plastic, in particular for improved cleaning and/or for improving the wearing comfort of textiles after washing, preferably LSEP plastic textiles, in particular textiles made of polyester or with a polyester content (blended fabric), or copolymers thereof.

17. The detergent or cleaning agent according to claim 14, wherein the peptide is conjugated to another molecule.


SEQUENCE LISTING (55 sequences)

SEQ ID NO: 1 - protein, 15 aa, synthetic construct
  REGION 1..15: note = Peptide RAL-M
RALQALRALQALEAL

SEQ ID NO: 2 - protein, 15 aa, synthetic construct
  REGION 1..15: note = Peptide RAL2E
RALRALRALEALEAL

SEQ ID NO: 3 - protein, 15 aa, synthetic construct
  REGION 1..15: note = Peptide RAL2Q
RALRALRALQALQAL

SEQ ID NO: 4 - protein, 15 aa, synthetic construct
  REGION 1..15: note = Peptide RALQE
RALRALRALQALEAL

SEQ ID NO: 5 - protein, 15 aa, synthetic construct
  REGION 1..15: note = Peptide RALQ2E
RALRALQALEALEAL

SEQ ID NO: 6 - protein, 17 aa, synthetic construct
  REGION 1..17: note = Peptide RAL2FQ
RALFEALQALFRALEAL

SEQ ID NO: 7 - protein, 14 aa, synthetic construct
  REGION 1..14: note = Peptide RALEQE
RALRALEALQALEA

SEQ ID NO: 8 - protein, 15 aa, synthetic construct
  REGION 1..15: note = Peptide RAL2FR
RALFEALFRALEALR

SEQ ID NO: 9 - protein, 14 aa, synthetic construct
  REGION 1..14: note = Peptide RAL2F
RALFEALFRALEAL

SEQ ID NO: 10 - protein, 13 aa, synthetic construct
  REGION 1..13: note = Peptide RALF
RALEALFRALEAL

SEQ ID NO: 11 - protein, 13 aa, synthetic construct
  REGION 1..13: note = Peptide RALF_sym
RALRALFEALEAL

SEQ ID NO: 12 - protein, 16 aa, synthetic construct
  REGION 1..16: note = Peptide RALF_Q
RALEALFRALQALEAL

SEQ ID NO: 13 - protein, 16 aa, synthetic construct
  REGION 1..16: note = Peptide RALFQ_W
RALEALWRALQALEAL

SEQ ID NO: 14 - protein, 13 aa, synthetic construct
  REGION 1..13: note = Peptide RALF_W
RALEALWRALEAL

SEQ ID NO: 15 - protein, 16 aa, synthetic construct
  REGION 1..16: note = Peptide RALA4Q
RALARALARALAQALA

SEQ ID NO: 16 - protein, 15 aa, synthetic construct
  REGION 1..15: note = Peptide P8_S2
RSIVTFSLRQNAQLA

SEQ ID NO: 17 - protein, 15 aa, synthetic construct
  REGION 1..15: note = PeptideP8_S8
RSIVTFSLRQNSEQA

SEQ ID NO: 18 - protein, 12 aa, synthetic construct
  REGION 1..12: note = Peptide BA1-A06
GLHTSATNLYLH

SEQ ID NO: 19 - protein, 12 aa, synthetic construct
  REGION 1..12: note = Peptide D2
QHSIRLLTIKKP

SEQ ID NO: 20 - protein, 12 aa, synthetic construct
  REGION 1..12: note = Peptide D4
QQSIRIMTIKHP

SEQ ID NO: 21 - protein, 12 aa, synthetic construct
  REGION 1..12: note = Peptide P3
WRHPRLRCGNLL

SEQ ID NO: 22 - protein, 12 aa, synthetic construct
  REGION 1..12: note = Peptide P14
QKSRNRMTRTHP

SEQ ID NO: 23 - protein, 12 aa, synthetic construct
  REGION 1..12: note = Peptide P6
SRARLFVVTYHK

SEQ ID NO: 24 - protein, 12 aa, synthetic construct
  REGION 1..12: note = Peptide P7
HMISTMNAASRR

SEQ ID NO: 25 - protein, 12 aa, synthetic construct
  REGION 1..12: note = Peptide P8
RSIVTFSLRQNR

SEQ ID NO: 26 - protein, 12 aa, synthetic construct
  REGION 1..12: note = Peptide P13
RNTIRIRTIKHP

SEQ ID NO: 27 - protein, 12 aa, synthetic construct
  REGION 1..12: note = Peptide P15
RHSSTLRYRPLP

SEQ ID NO: 28 - protein, 16 aa, synthetic construct
  REGION 1..16: note = Peptide RAL-M-C
RALQALRALQALEALC

SEQ ID NO: 29 - protein, 16 aa, synthetic construct
  REGION 1..16: note = Peptide RAL2E-C
RALRALRALEALEALC

SEQ ID NO: 30 - protein, 16 aa, synthetic construct
  REGION 1..16: note = Peptide RAL2Q-C
RALRALRALQALQALC

SEQ ID NO: 31 - protein, 16 aa, synthetic construct
  REGION 1..16: note = Peptide RALQE-C
RALRALRALQALEALC

SEQ ID NO: 32 - protein, 16 aa, synthetic construct
  REGION 1..16: note = Peptide RALQ2E-C
RALRALQALEALEALC

SEQ ID NO: 33 - protein, 18 aa, synthetic construct
  REGION 1..18: note = Peptide RAL2FQ-C
RALFEALQALFRALEALC

SEQ ID NO: 34 - protein, 15 aa, synthetic construct
  REGION 1..15: note = Peptide RALEQE-C
RALRALEALQALEAC

SEQ ID NO: 35 - protein, 16 aa, synthetic construct
  REGION 1..16: note = Peptide RAL2FR-C
RALFEALFRALEALRC

SEQ ID NO: 36 - protein, 15 aa, synthetic construct
  REGION 1..15: note = Peptide RAL2F-C
RALFEALFRALEALC

SEQ ID NO: 37 - protein, 14 aa, synthetic construct
  REGION 1..14: note = Peptide RALF-C
RALEALFRALEALC

SEQ ID NO: 38 - protein, 14 aa, synthetic construct
  REGION 1..14: note = Peptide RALF_sym
RALRALFEALEALC

SEQ ID NO: 39 - protein, 17 aa, synthetic construct
  REGION 1..17: note = Peptide RALF_Q-C
RALEALFRALQALEALC

SEQ ID NO: 40 - protein, 17 aa, synthetic construct
  REGION 1..17: note = Peptide RALFQ_W-C
RALEALWRALQALEALC

SEQ ID NO: 41 - protein, 14 aa, synthetic construct
  REGION 1..14: note = Peptide RALF_W-C
RALEALWRALEALC

SEQ ID NO: 42 - protein, 17 aa, synthetic construct
  REGION 1..17: note = Peptide RALA4Q-C
RALARALARALAQALAC

SEQ ID NO: 43 - protein, 16 aa, synthetic construct
  REGION 1..16: note = Peptide P8_S2-C
RSIVTFSLRQNAQLAC

SEQ ID NO: 44 - protein, 16 aa, synthetic construct
  REGION 1..16: note = Peptide P8_S8-C
RSIVTFSLRQNSEQAC

SEQ ID NO: 45 - protein, 13 aa, synthetic construct
  REGION 1..13: note = Peptide BA1-A06-C
GLHTSATNLYLHC

SEQ ID NO: 46 - protein, 13 aa, synthetic construct
  REGION 1..13: note = Peptide D2-C
QHSIRLLTIKKPC

SEQ ID NO: 47 - protein, 13 aa, synthetic construct
  REGION 1..13: note = Peptide D4-C
QQSIRIMTIKHPC

SEQ ID NO: 48 - protein, 13 aa, synthetic construct
  REGION 1..13: note = Peptide P3-C
WRHPRLRCGNLLC

SEQ ID NO: 49 - protein, 13 aa, synthetic construct
  REGION 1..13: note = Peptide P14-C
QKSRNRMTRTHPC

SEQ ID NO: 50 - protein, 13 aa, synthetic construct
  REGION 1..13: note = Peptide P6-C
SRARLFVVTYHKC

SEQ ID NO: 51 - protein, 13 aa, synthetic construct
  REGION 1..13: note = Peptide P7-C
HMISTMNAASRRC

SEQ ID NO: 52 - protein, 13 aa, synthetic construct
  REGION 1..13: note = Peptide P8-C
RSIVTFSLRQNRC

SEQ ID NO: 53 - protein, 13 aa, synthetic construct
  REGION 1..13: note = Peptide P13-C
RNTIRIRTIKHPC

SEQ ID NO: 54 - protein, 13 aa, synthetic construct
  REGION 1..13: note = Peptide P15-C
RHSSTLRYRPLPC

SEQ ID NO: 55 - protein, 12 aa, synthetic construct
  REGION 1..12: note = Peptide MS-S1
ATIHDAFYSAPE